(19) United States
(12) Patent Application Publication
(10) Pub. No.: US 2014/0113361 A1
(43) Pub. Date: Apr. 24, 2014

(54) PLANT FOR PRODUCTION OF BIOGAS

(71) Applicant: Armando Berasi, Trento (IT)

(72) Inventor: Armando Berasi, Trento (IT)

(21) Appl. No.: 14/115,850

(22) PCT Filed: May 7, 2012
(86) PCT No.: PCT/IB2012/052262

§ 371 Date: Dec. 11, 2013

(30) Foreign Application Priority Data
May 6, 2011 (IT) VR2011A000095

(51) Int. Cl. C12M 1/00

(52) U.S. Cl. CPC C12M 21/12
USPC 435/289.1

(57) ABSTRACT

The present disclosure refers to a plant (1) for production of biogas (18). The plant (1) comprises a digester (4) including a bag-like casing (5) of flexible material and fluid-tight, wherein the bag-like casing (5) is adapted for the containing a sewage (14). The casing (5) defines, in an internal zone, a digestion chamber (40) adapted for a digestion of the sewage (14). The plant (1) further comprises a heater (45, 68) for heating the sewage (14). The disclosure further refers to a co-generation installation (100) comprising a plant (1) for the production of biogas (18), a co-generator (8) and fuelling means (71, 72) for fuelling the co-generator (8) with biogas (18), said fuelling means comprising a biogas line (71, 72) between the plant (1) and the co-generator (8).

The present disclosure refers to a plant for production of biogas. To be more specific, it refers to a plant for the production of biogas via digestion of sewage, which in particular is a liquid wastewater originating from a zootechnic farm. An analogous plant can be used also for the digestion of a wastewater coming from sewers.

In the art some types of plants for the production of biogas are known.

A first type of plants provides for a first digester and a second digester, which are closed tanks having a vertical development and made of reinforced concrete.

The first digester is fed with a substrate consisting of a mixture of solid biomass (which usually is corn silage or another silage) and sewage, which are mixed in a pumping and mixing station. The first digester starts the substrate digestion; the partially-digested substrate in the first digester is transferred to the second digester, where the digestion phase is completed. The biogas produced by the digestion is extracted from both digesters and is stored or used as fuel by a co-generator.

This first type of plants requires substantial construction works for building the reinforced-concrete digesters, the loading ramps for the solid substance and the stocking trenches for the solid substance. Moreover, due to the height of the digesters, the plant has a marked visual and environmental impact.

A second type of plants provides for a first tube-shaped horizontal digester and a second vertical digester which has a reduced size compared to the digesters belonging to the first type of plants. The second digester can be made of reinforced concrete or steel.

In these plants, the feeding substrate of the first digester has a solid biomass content which is very high in comparison with the sewage content.

Hydrolysis of the organic substance under mesophilic conditions takes place in the first digester; the partially-digested substrate is transferred to the second digester, which works under thermophilic conditions.

The biogas produced by the digestion is extracted from both digesters and is stored or used as fuel by a co-generator.

Also the second type of plants requires substantial construction works, which are needed for building the digesters and the stocking trenches for the biomass.

A common problem of the known plants is that, due to the required construction works, they entail a considerable financial investment for their building.

Moreover, expensive and impacting materials, as concrete and steel, are used to build the known plants.

As a consequence, in order to have an acceptable investment-return period, the known plants are usually fed with a substrate having a high content of solid biomass (for example, corn silage) so as to have a high biogas production, which ensures a financial return thanks to electric energy production via biogas combustion in a co-generator.

As an indicative example, it should be considered that the digestion of 1 cubic meter of sewage produces 30 cubic meters of biogas, whereas the digestion of 1 cubic meter of corn silage produces 150 cubic meters of biogas.

It is therefore apparent that the known plants are used trying to maximise the content of “valuable” solid substance (i.e., capable of turning into a high amount of produced biogas) and thus the plant yield.

Moreover, the required investment cost poses serious constraints when designing a known plant.

For example, referring to a zootechnic farm having a certain number of head and a specific sewage production, the digestion plant cannot be designed on the actual amount of available sewage, due to the high investment cost: if the plant were designed on the actual available sewage, it would have a prohibitive cost compared to the farm budget.

Therefore, the known plants are usually undersized compared to the actual availability of sewage and they are fed also with valuable solid biomass in order to increase the biogas production and the financial performance.

Basically, the known plants are not suitable for an effective exploitation of zootechnic sewage. The zootechnic sewage, even if available in a huge amount and at a minimal cost, can be exploited only in part, whereas the part which is not exploited has to be disposed bearing the consequent costs and environmental impacts.

Some digestion system provides for using flexible digesters. For example, international patent application No. WO 2009/073902 A2 discloses a biomass digester system including flexible digesters having an elongated shape.

U.S. Pat. No. 7,186,339 discloses an anaerobic digester system which includes a substantially flexible bladder.

International patent application No. WO 02/062497 A1 discloses a system wherein a mixture of animal waste is treated and inserted into a plastic bag.

U.S. Pat. No. 5,080,786 discloses a biomass digester having an air-tight structure which can be collapsed under atmospheric pressure.

U.S. Pat. No. 4,100,023 discloses a digester for organic matter having a flexible chamber.

U.S. Pat. No. 4,579,654 discloses an apparatus for anaerobic fermentation, made of a flexible gas-tight material.

US patent application No. US 2010/284749 discloses a flexible digester which however, being intended to work underwater, has an application field which is different from the field of the present disclosure.

These known plants having a flexible digester may, in case, allow to simplify the building of the plant, in particular in relation to the construction works and the materials to be used. However, they show drawbacks in view of the digestion process management, the digestion efficiency and the yield.

The present disclosure therefore moves from the technical problem of providing a plant for the production of biogas which allows to overcome the drawbacks mentioned above with respect to the prior art and/or which is able to achieve further advantages.

This is obtained by providing a plant for the production of biogas according to independent claim 1. A subject of the present disclosure is also a co-generation installation according to claim 15.

Secondary features of the subject of the present disclosure are defined in the corresponding depending claims.

Basically, a plant for the production of biogas according to the present disclosure includes a digester having a digestion chamber which is enclosed and bounded by a casing of flexible material and fluid-tight.

In other words, the casing is a bag or balloon which, being made of a flexible material, can take a loose or collapsed configuration, and a bloated or stretched configuration.

The casing is adapted to contain sewage to be digested, therefore the digestion of sewage takes place inside the casing, i.e. in the digestion chamber defined by the casing itself. The walls of the casing are fluid-tight (where both a liquid substance as sewage and a gaseous substance as biogas are meant by “fluid”) and then biogas produced by the digestion stays inside the casing, gathering in a top region of the latter, from which it is taken out through suitable extraction means.

The building of the digester is simpler and less expensive than the known art, because the construction works to be done are strongly reduces and the use of expensive materials is avoided as much as possible. For example, no construction works of reinforced concrete or iron are required for building the digester.

The main body of the digester, which in some known plants is a tank or a cylindrical body of reinforced concrete or metal, is here substantially a “balloon” or “bag” of flexible material, which is prefabricated and then installed in short times, since it requires a minimal amount of labour. For example, in some embodiments, the casing or bag rests directly on a ground, in such a way that a bottom face of the casing rests on a surface of a compartment or basin, i.e. on a resting surface for the digester.

Thanks to the lightness of the material of the casing, which for example is made of nylon fabric laminated to two layers of PVC, and to the small thickness (for example, 4 mm) which is required for the casing walls, the production cost is much lower in comparison with the known digesters.

Thanks to the reduced cost per cubic meter of digestion chamber, the building of a digester having a large volume, which is able to process a large amount of sewage also making acceptable a yield lower than the known plant, is profitable. For example, in some embodiment, the digestion chamber has a volume greater than 3000 cubic meters, in particular 5000 cubic meters.

A plant for the production of biogas according to the present disclosure is useful for allowing an exploitation of a greater amount of organic wastewater, making profitable also the exploitation of those amounts which, otherwise, would not be exploited by the known plants.

A plant according to the present disclosure is useful also for producing biogas without necessarily resorting to supplements to the sewage through “valuable” biomass.

For example, in some embodiment the feeding sewage, with which the digester is fed, has an organic substance concentration which is lower than 15% by weight, i.e. it is a substantially “natural” sewage to which no solid biomass has been added to increase its specific organic content.

A plant according to the present disclosure is useful for making a plant which is simpler and cheaper than the known plants and which allows to process a large amount of sewage at a low cost.

In some embodiments, the bag (and than the plant) has a substantially horizontal development, i.e. the bag has a height which is substantially lower than the length and the width of the bag itself. In other words, the bag takes up a remarkable surface in a plan view, but it has a development in height which is very low. In particular, if the bag is partially underground, it slightly sticks out from the ground (for example, one or two meters).

This is useful for having a digester with a visual, environmental and landscape impact which is reduced to a minimum or even eliminated.

In some embodiments, the bag is at least partially underground in a pit or trench, so that a bottom face of the bag rests on a bottom surface of the pit, and a lateral face of the bag rests at least partially on a lateral surface of the pit.

This is useful for having a bag that is firmly placed in the ground and that does not change its shape depending on the load of sewage inside the bag. Furthermore, the pit acts as a containment and protection for the bag.

For example, during the working of the plant, the free surface of the sewage in the digestion chamber is kept at a level lower than the plane of site, i.e. the sewage level in the digestion chamber is lower than the depth of the pit in which the bag is housed. This is useful for ensuring that the weight of the sewage in the bag is entirely discharged on the walls of the pit and does not affect the bag region which, emerging from the ground, may deform under the weight of the liquid (for example, in the case where the ground surrounding the bag is not flat).

In some embodiments, a heat insulating cloth or the like is sandwiched between the bag and the ground; this is useful for reducing the heat loss from the digestion chamber to the ground, so that it is easier to maintain a temperature suitable to the digestion process in the digestion chamber.

Furthermore, a sewage heater inside the digestion chamber is provided, in order to adjust the temperature of the sewage to be digested, and to ensure a good production yield of biogas.

For example, the heater comprises a heat exchanger, outside the bag, heating a recirculated sewage portion, i.e. sewage taken from the digestion chamber and re-introduced into the digestion chamber after the heating. This is useful for an easily controllable heating, having good heat exchange coefficients, in other words for an effective and precise control of the temperature in the digestion chamber.

In some embodiments, the sewage is re-introduced into the digestion chamber at the sewage surface and/or tangentially to the lateral walls of the bag, so as to create mixing flows of the sewage in the digestion chamber. This is useful for a better homogenisation of the sewage during the digestion and also to perform a cleaning action on the inner walls of the bag, as well as to remove and prevent stagnation of sewage along the walls and in the corners of the bag.

The heat for the heater operation can be recovered from the cooling of a co-generator fuelled with the biogas produced by the plant. This is useful for increasing the overall energy yield of the plant.

The heater can be or can comprise also a coil in which a fluid circulates, for example water, which is heated in a heating system such as a boiler or a heat recovery system; in some embodiments, the coil is positioned inside the bag, i.e. directly in the digestion chamber, whereas in other embodiments the coil is sandwiched between the bag and the ground (in particular, between the bag and the heat insulating cloth).

In one embodiment, the plant comprises a second bag which receives sewage from the first bag and carries out a second digestion phase. In other words, the first bag and the second bag compose a digester which works according to two digestion phases in a series. This is useful for increasing the yield of the plant, lengthening the time of residence, increasing the production of biogas and the quality of the latter.

In one embodiment, a return line allows to transfer sewage from the second bag to the first bag, thus carrying out a dilution of the fresher sewage by an at least partially digested sewage. This is useful for controlling the nitrogen load, i.e. for optimally managing the concentration of nitrogen in the digestion chamber of the first bag, preventing the latter from having a high nitrogen concentration that could stop the digestion.

In one embodiment, a part of the biogas taken from the plant is re-introduced into the bag (and/or in the second bag, if available), when the instant production of biogas exceeds the feed that can be burned by the co-generator at the time. For this purpose, a pressure relief valve on the biogas piping and a return piping connecting the pressure relief valve to the bag are provided. This is useful for managing the working of the co-generator at the best, avoiding overloads of the latter and trying to keep a working regime as uniform as possible. In other words, the bag is also used as temporary storage of biogas, allowing also a homogenisation of the quality of biogas produced during a day.

Further advantages, characteristic features and the modes of use of the subject of the present disclosure will become clear from the following detailed description of a preferred embodiment thereof, provided by way of example and not for limitative purposes.

It is clear, however, that each embodiment of the subject of the present disclosure may have one or more of the advantages listed above; in any case it is not required that each embodiment has simultaneously all the advantages listed.

Reference shall be made to the figures in the accompanying drawings in which:

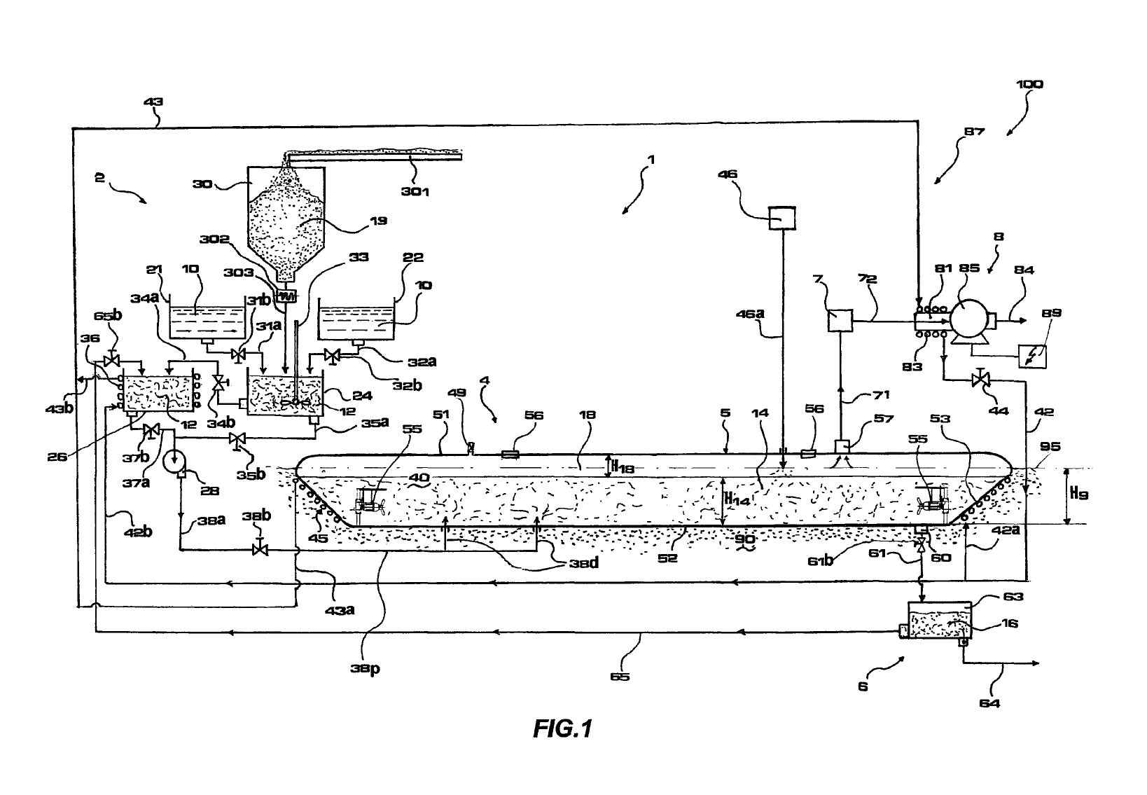

FIG. 1 shows a schematic side view of a first embodiment of a plant for biogas production according to the present disclosure;

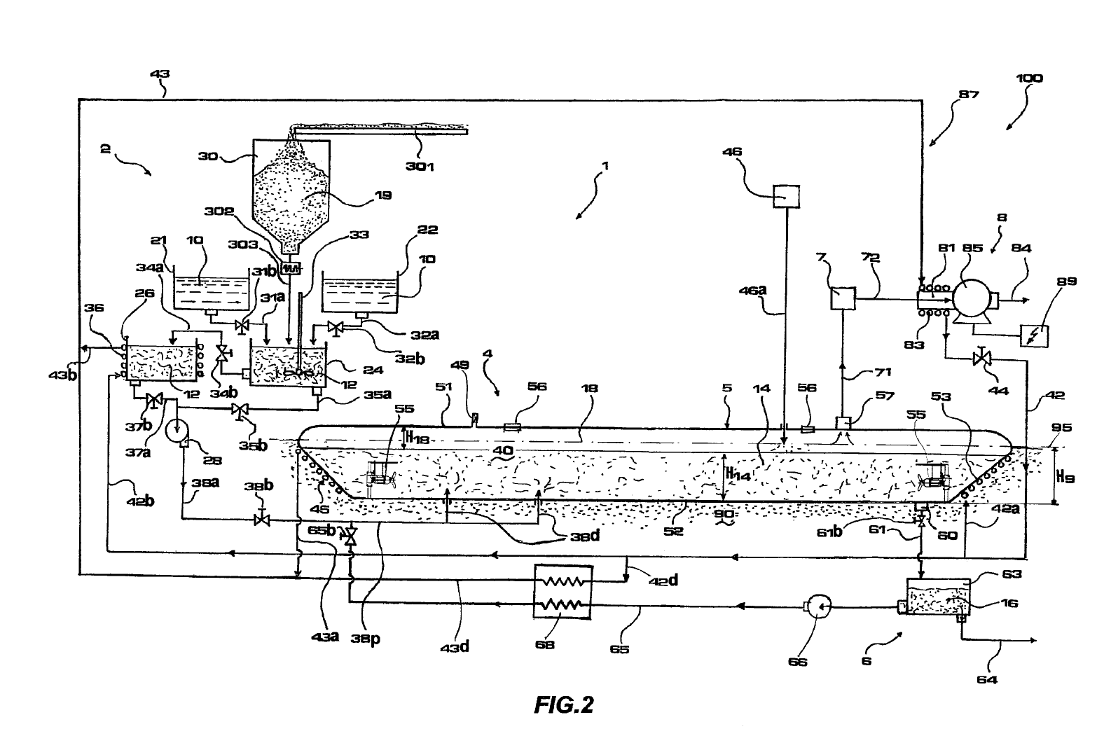

FIG. 2 shows a schematic side view of a second embodiment of a plant for biogas production according to the present disclosure;

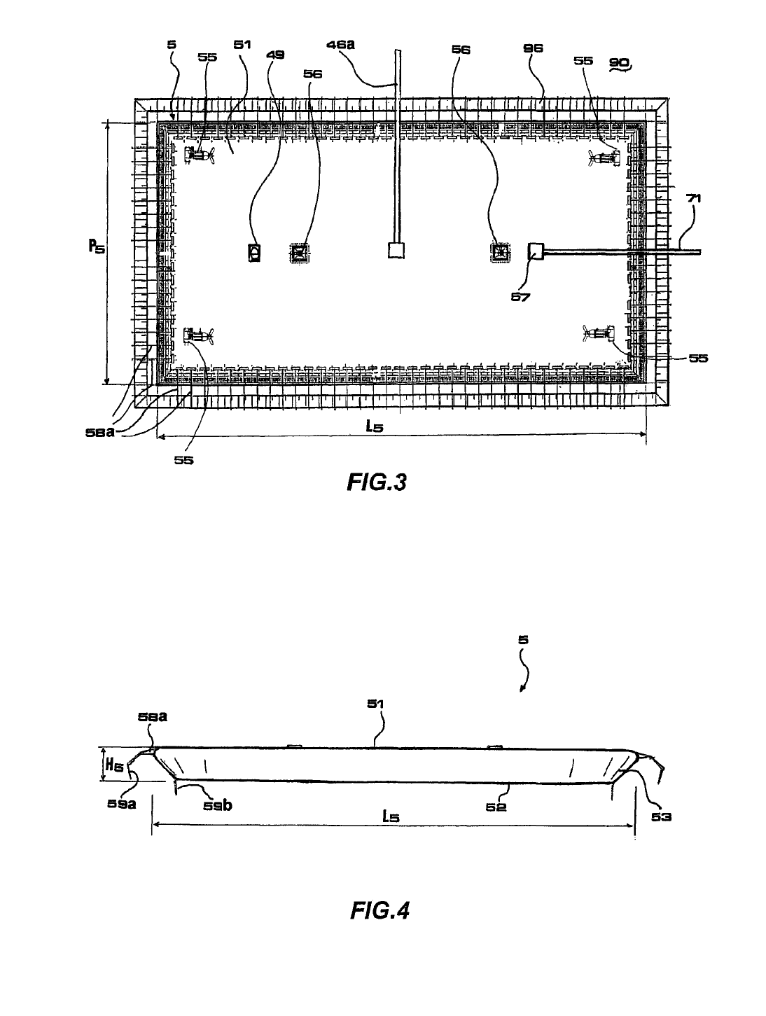

FIG. 3 shows a top view of a digester of a plant for biogas production according to the present disclosure;

FIG. 4 shows a side view of a component of the digester of FIG. 3;

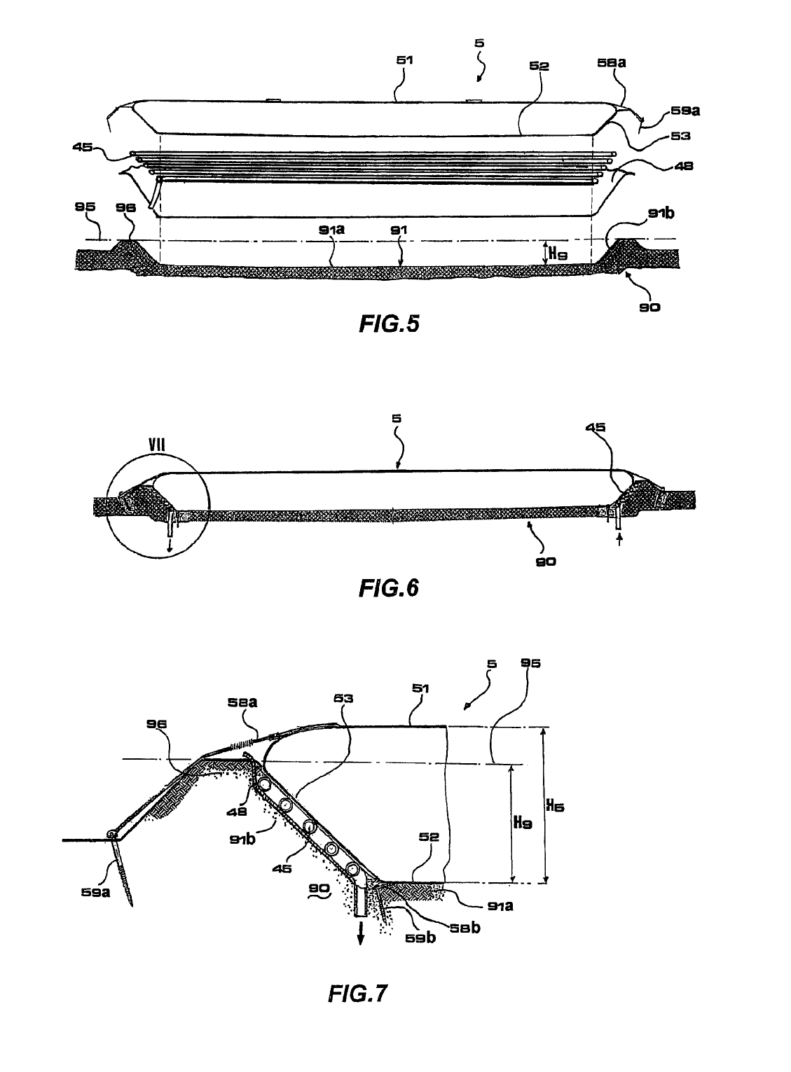

FIG. 5 shows a partially-sectional side view, with separated parts, of a digester according to the present disclosure, from which some parts have been removed;

FIG. 6 shows a partially-sectional side view of the digester of FIG. 5;

FIG. 7 shows an enlarged view of a detail VII of FIG. 6;

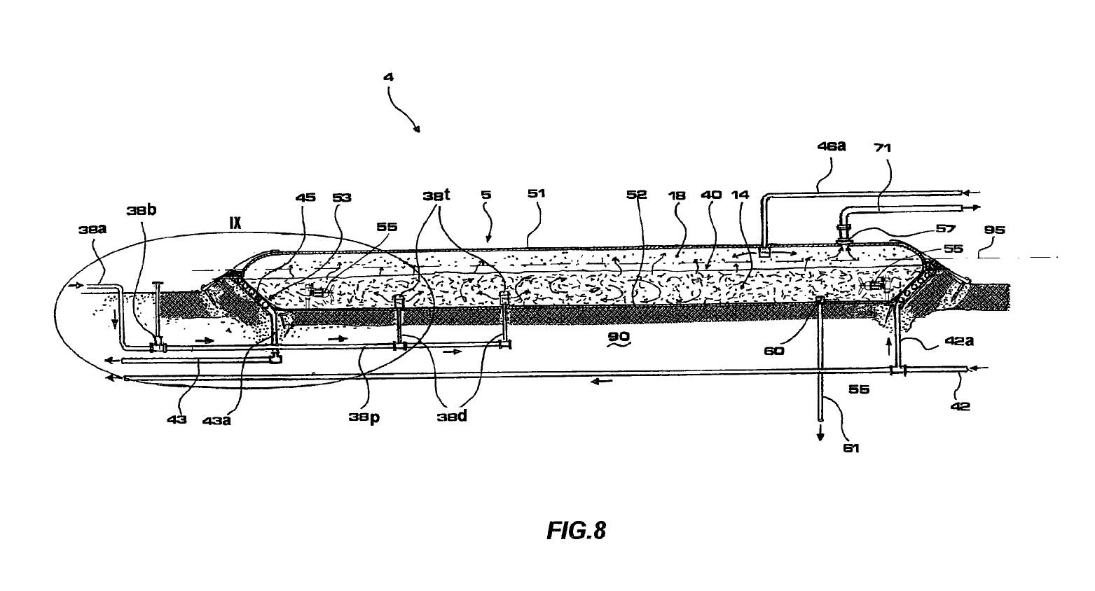

FIG. 8 shows a sectional side view of a digester according to the present disclosure, in an operating condition;

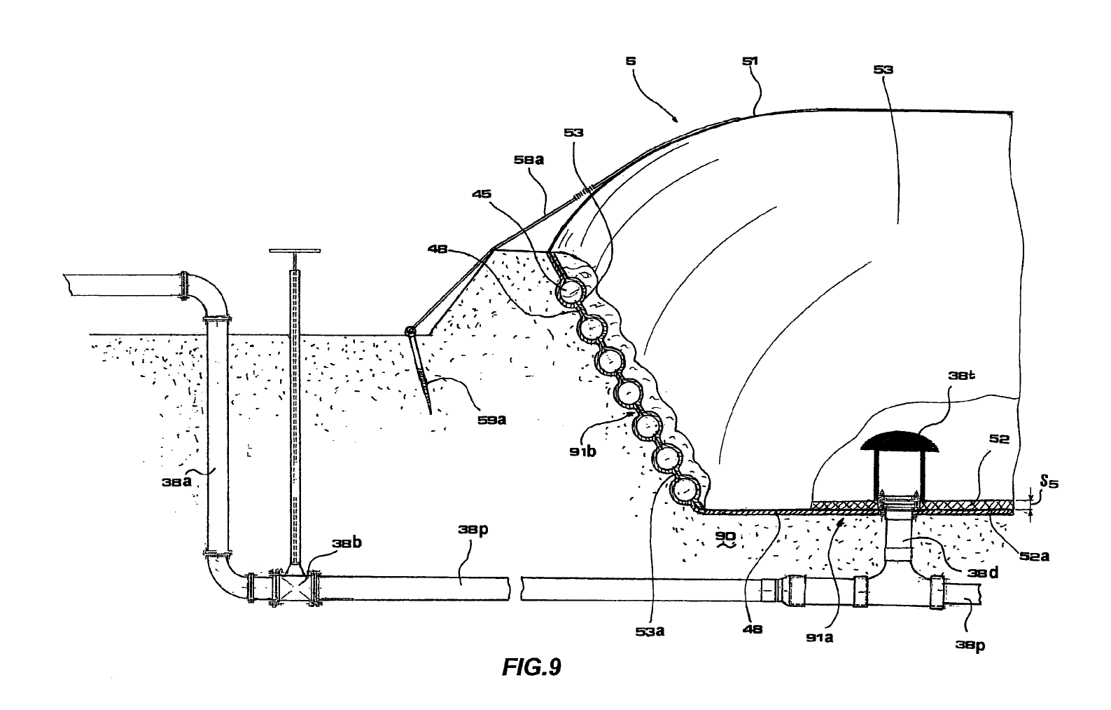

FIG. 9 shows a partially-sectional enlarged view of a detail IX of FIG. 8;

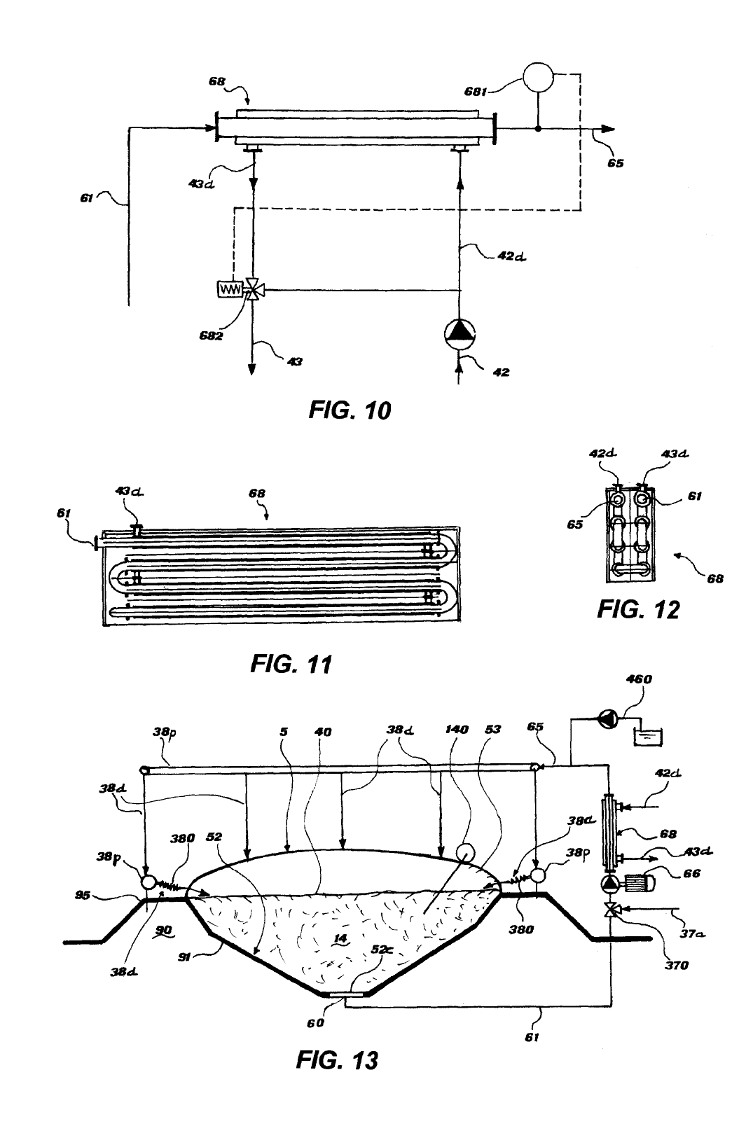

FIG. 10 shows a schematic view of a heater for a plant for biogas production according to the present disclosure;

FIG. 11 shows a partially-sectional front view of the heater of FIG. 10;

FIG. 12 shows a partially-sectional side view of the heater of FIG. 10;

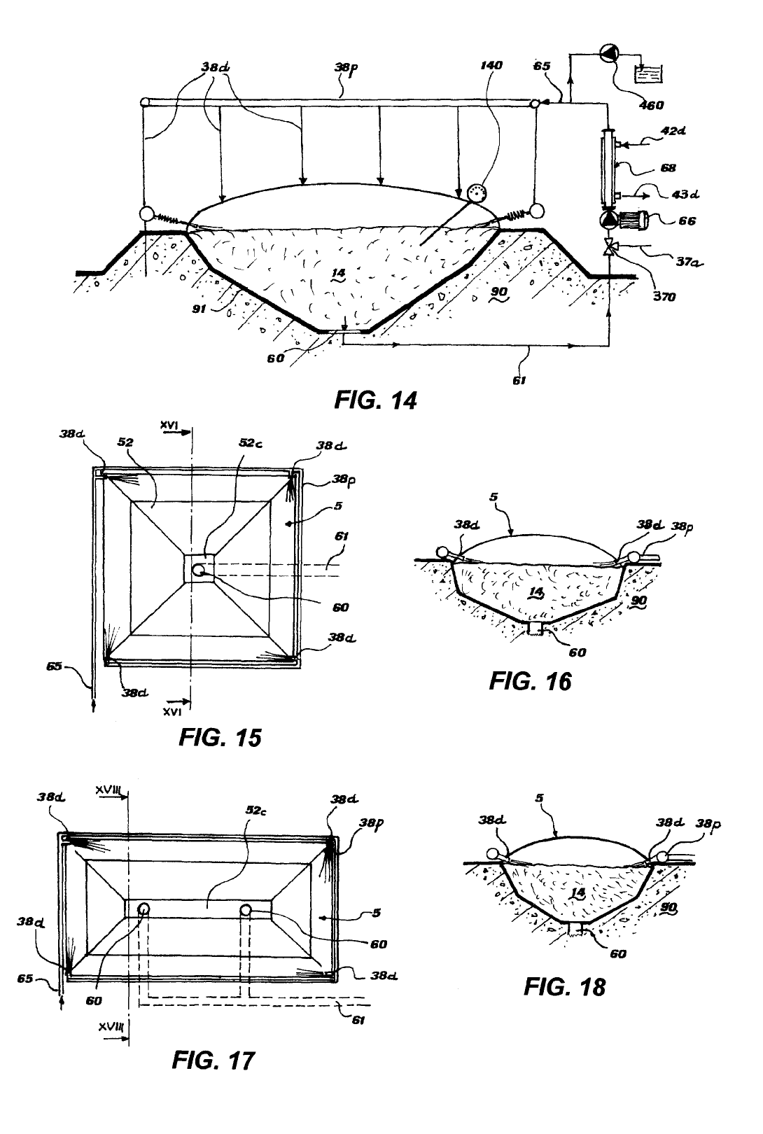

FIGS. 13 and 14 schematically show a sectional view of an embodiment of a digester according to the present disclosure, in two operating phases;

FIG. 15 shows a top view of the inside of a digester according to the present disclosure, from which some parts have been removed;

FIG. 16 shows a sectional side view, according to a section line XVI-XVI, of the digester of FIG. 15;

FIG. 17 shows a top view of the inside of another digester according to the present disclosure, from which some parts have been removed;

FIG. 18 shows a sectional side view, according to a section line XVIII-XVIII, of the digester of FIG. 17;

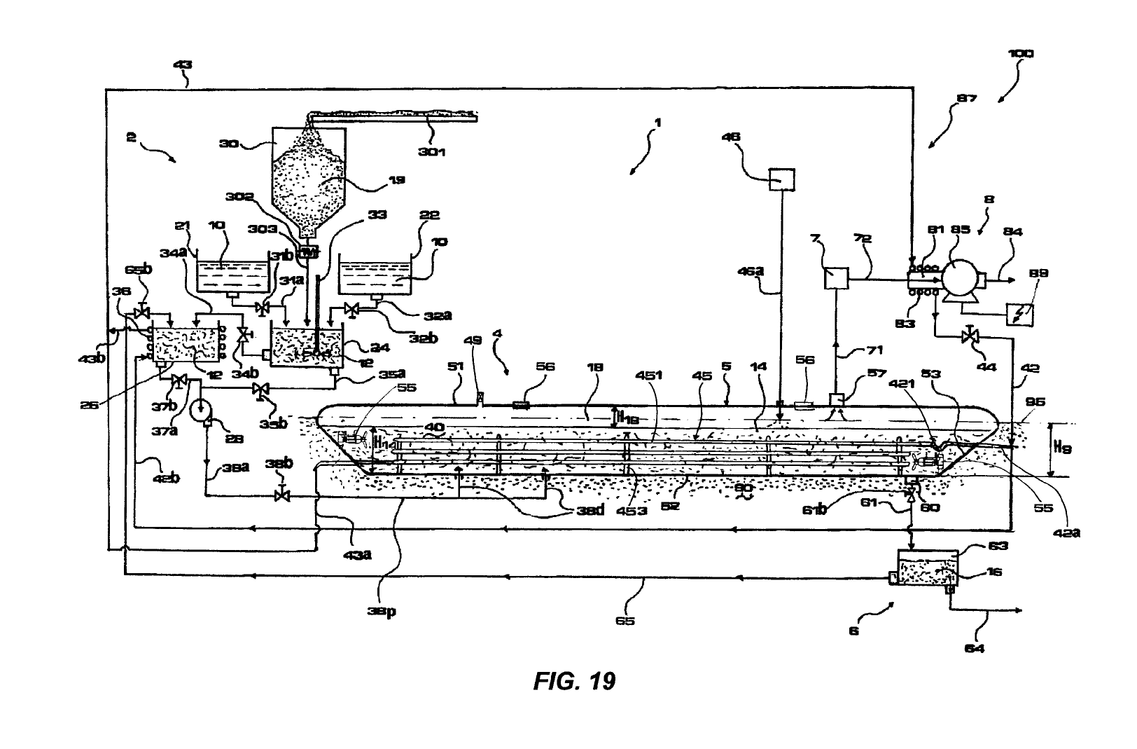

FIG. 19 shows a schematic side view of a third embodiment of a plant for biogas production according to the present disclosure;

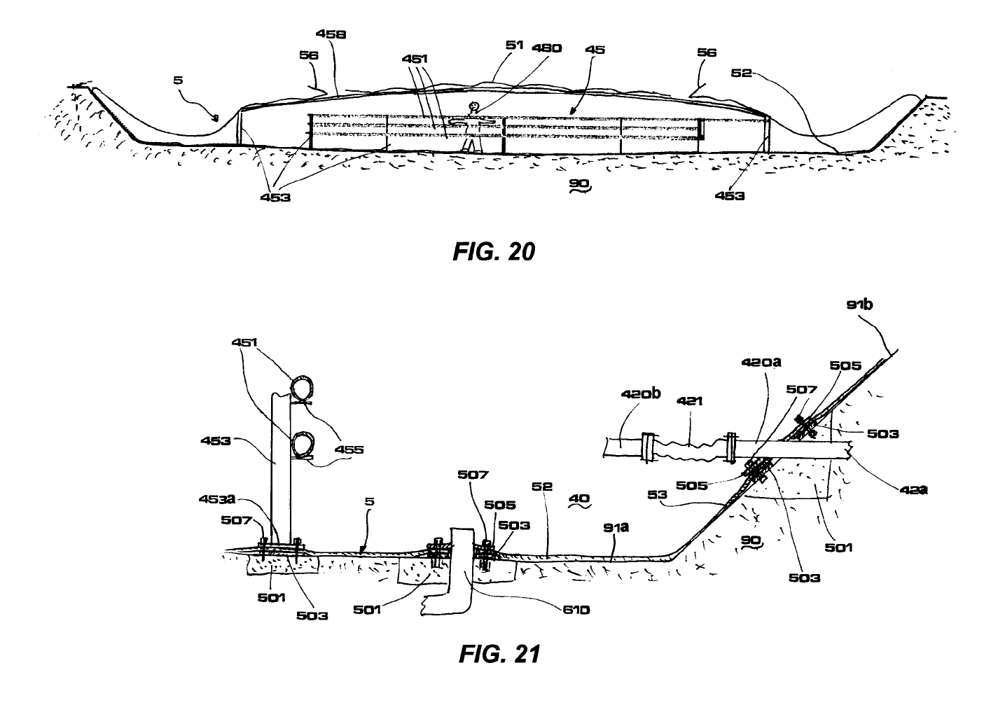

FIG. 20 shows a side view of the plant of FIG. 19 in a construction phase;

FIG. 21 shows a partially-sectional side view of a detail of FIG. 19, from which some parts have been removed;

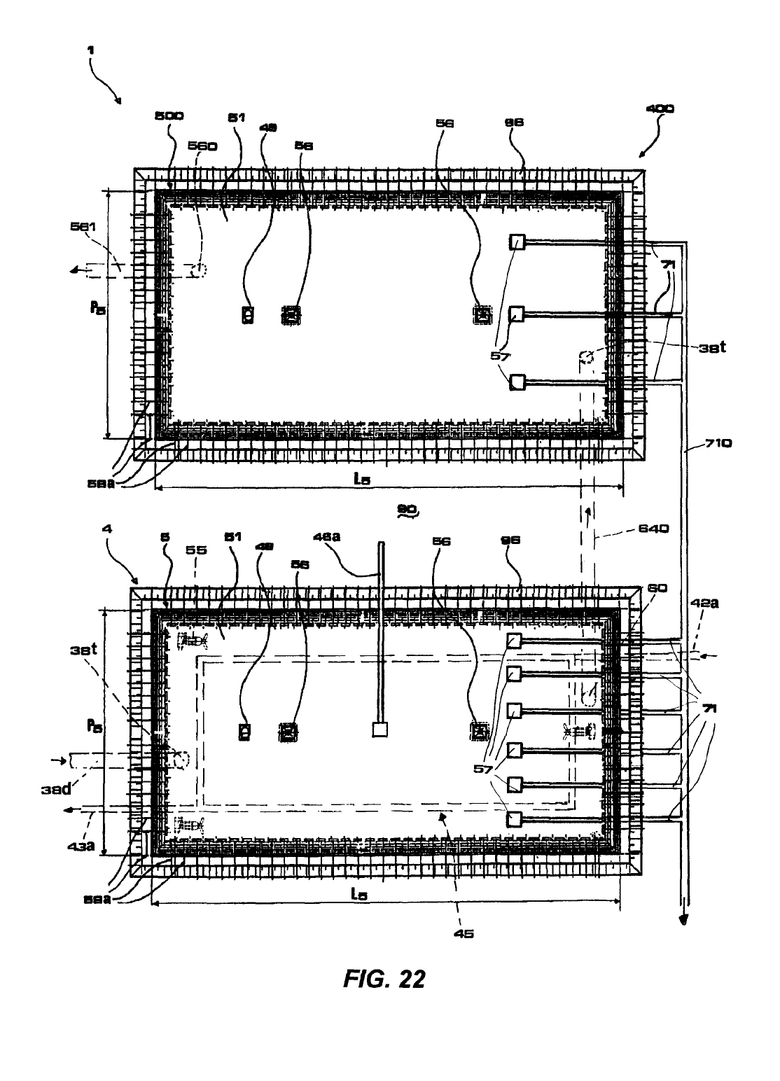

FIG. 22 shows a schematic top view of a fourth embodiment of a plant for biogas production according to the present disclosure;

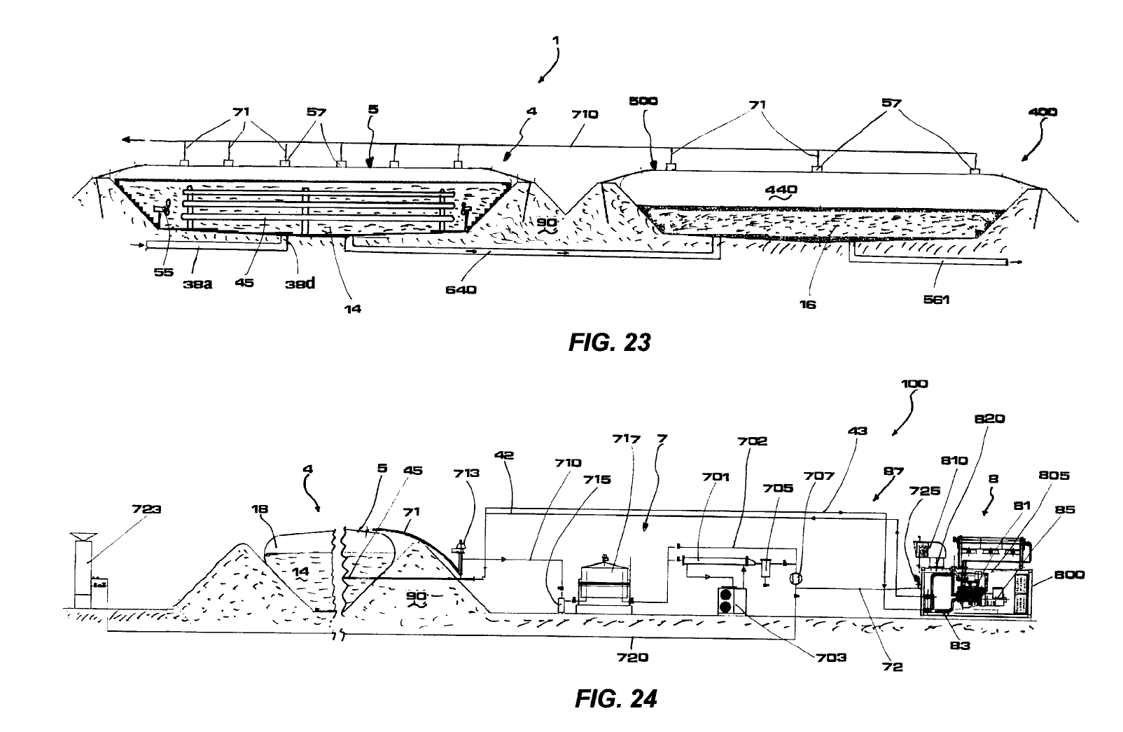

FIG. 23 shows a schematic side view of the plant of FIG. 22;

FIG. 24 shows a schematic side view of a co-generation installation according to the present disclosure;

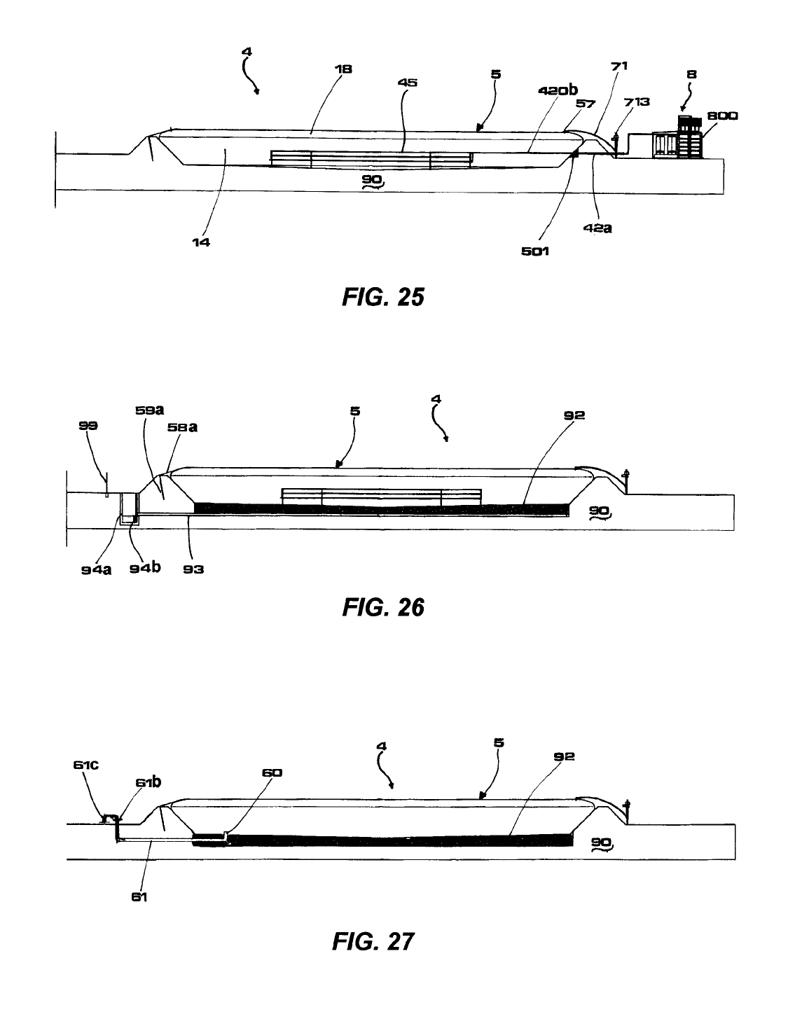

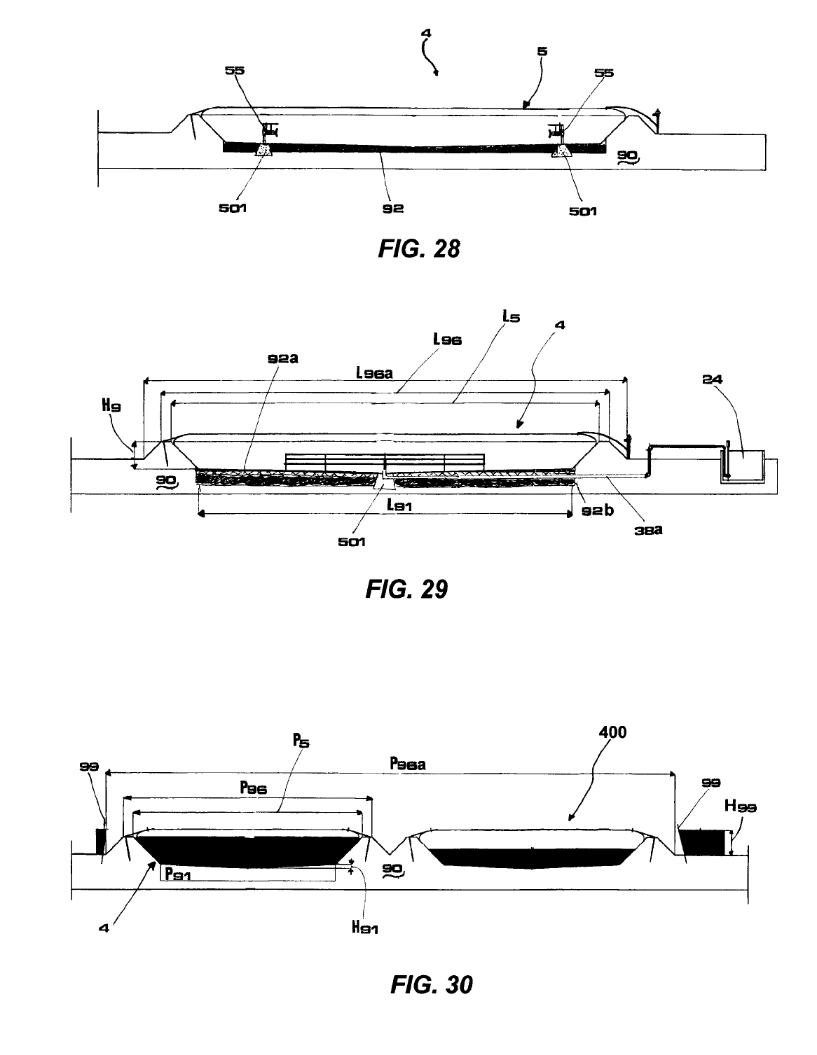

FIGS. 25 to 29 show schematic side views of the plant of FIG. 19, in each of which some parts have been removed to highlight specific aspects;

FIG. 30 shows another schematic side view of the plant of FIG. 22;

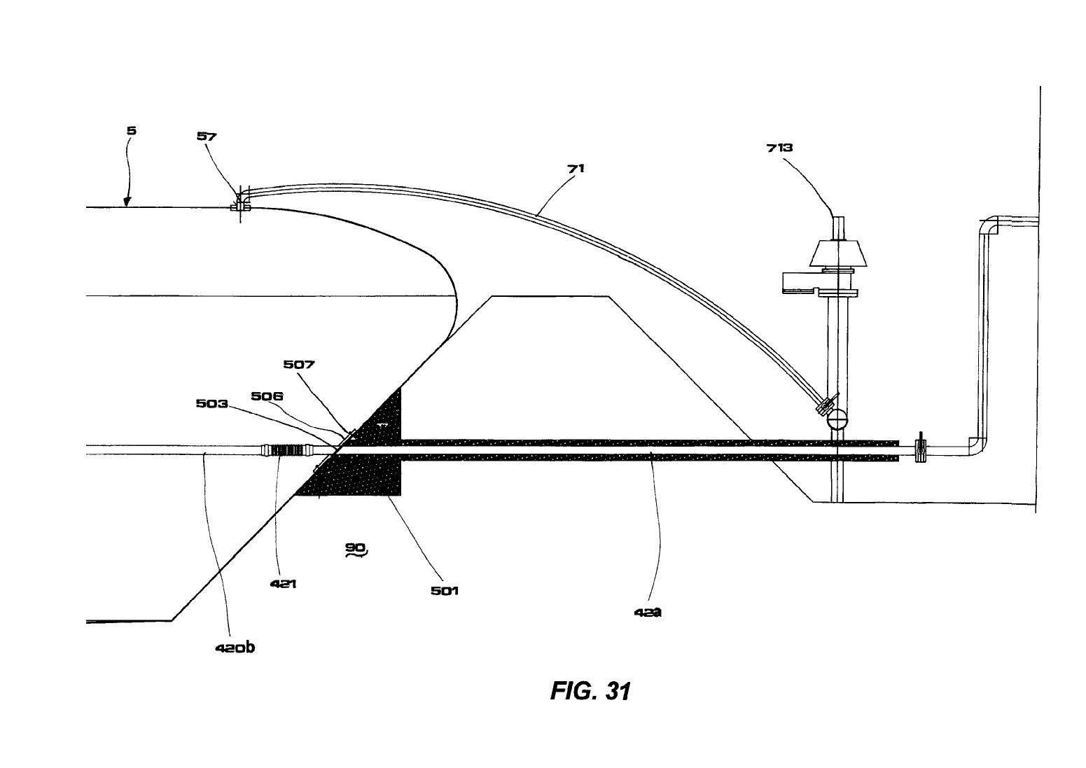

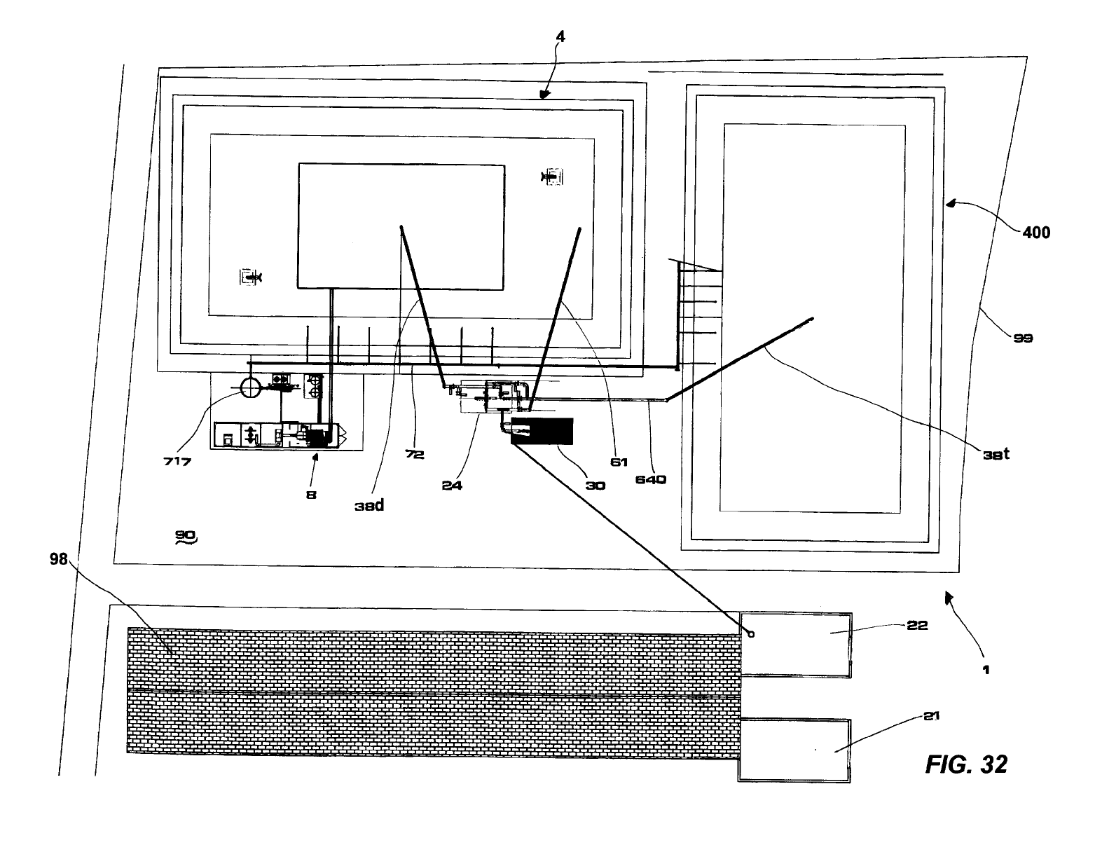

FIG. 31 shows a different version of FIG. 21;

FIG. 32 shows another schematic top view of the plant of FIG. 22;

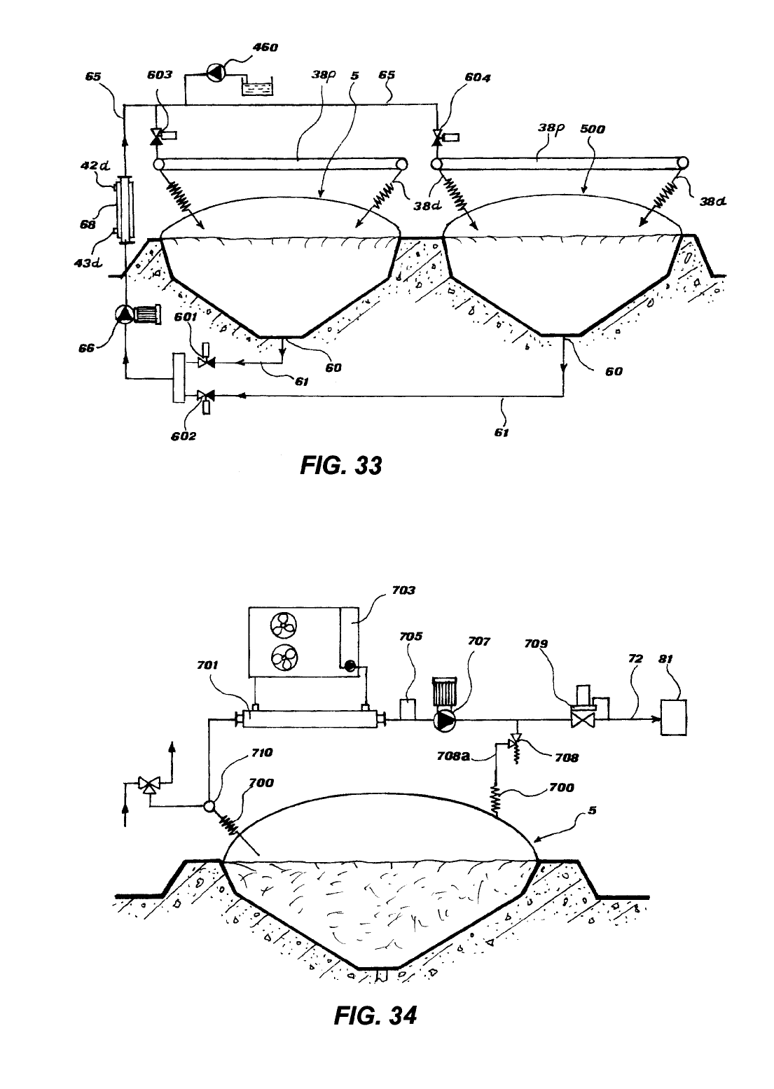

FIG. 33 shows a schematic sectional side view of a further embodiment of a plant for biogas production according to the present disclosure;

FIG. 34 shows a schematic sectional side view of a different version of a portion of a plant according to the present disclosure.

Referring first to FIGS. 1 to 9, a plant for biogas production according to the present disclosure is denoted as a whole by reference number 1.

The plant 1 described below is configured to produce biogas 18 via digestion of a sewage 10 originating, for example, from a zootechnic farm.

As “zootechnic sewage”, it is meant an aqueous-based liquid containing organic or inorganic substances, in particular vegetal waste or waste substances or waste produced by animal metabolism, which are digestible by microorganisms. The organic or inorganic substances are dissolved in the liquid phase and/or are solid particles dispersed in the liquid phase.

The sewage 10 is “fresh”, i.e. it has not yet been treated; in other words, the sewage 10 is a feeding input to the plant 1 that has not yet been treated by the plant 1 itself.

The plant 1, schematically shown in FIG. 1, comprises:
a section 2 for preparation and feeding of the sewage to be digested;
a digester 4, which digests the sewage fed from section 2 and produces biogas;
a section 6 for treatment of the digested sewage;
a section 7 for conditioning the produced biogas.

In the example, the plant 1 is connected with a co-generator 8, which includes a combustion engine 81 and an electric generator 85 (for example an alternator or a dynamo) driven by the combustion engine 81. The combustion engine 81 is fuelled by the biogas produced by the plant 1 itself.

The combustion engine 81 is provided with a cooling circuit 83 with heat recovery; the heat recovery can be increased by providing a heat exchanger between the cooling circuit 83 and the exhaust pipe 84 of the engine 81, so as to recover also a part of the thermal energy of the exhaust gases.

Overall, the plant 1 for biogas production and the co-generator 8 can therefore be seen as a plant for the production of electric energy and/or thermal energy, i.e. a co-generation installation 100.

In a simplified embodiment, the preparation-and-feeding section 2 includes:
a tank for storage of sewage produced by the farm, in which a load pump is included;
a hopper with a system of extraction and weighing of the product, in order to add biomass to the sewage if a raising of the organic load is required;
a feed pump which carries out the mixing of the biomass/sewage; the feed pump is fed with various vegetal biomass products and sewage according to a programmed recipe book. This loading system allows to vary the recipe for feeding the digester as a function of the vegetal products which are wanted to be used. The feed pump transfers the homogenised sewage to the digester, where it mixes with a sewage that is already in digestion in the digester.

In the embodiment shown in FIG. 1, which is more complex than that indicated above, the preparation-and-feeding section 2 includes:
a first tank 21 and a second tank 22 for storage of sewage 10 produced by the farm (or fresh sewage);
a tank 24 for preparation or pre-loading;
a tank 26 for pre-heating;
a load or feed pump 28.

The first and second storage tanks 21, 22 are fed with the fresh sewage 10 that originates from a zootechnic farm. For example, the storage tanks 21, 22, or cisterns, have a capacity of 40 cubic meters each.

The storage tanks 21, 22 are connected, through respective pipes 31a, 32a, with the pre-load tank 24. The pipes 31a, 32a are provided with shut-off valves 31b, 32b and any pumps (not shown).

The pre-load tank 24 has, for example, a capacity of 8-10 cubic meters and is provided with a mixer 33 for mixing and homogenising the fresh sewage 10, to obtain a homogenised sewage 12 to be fed to the digester 4. The pre-load tank 24 is, for example, of stainless steel.

The preparation-and-feeding section 2 comprises a hopper 30 for adding biomass 19 to the sewage, when it is necessary to raise the organic load. The biomass 19 is for example a vegetal biomass as cereal flour, molasses, waste flour, green mowed waste, other digestible substances.

The hopper 30 is connected to the pre-load tank 24 through a pipe or channel 303. The hopper 30 can be equipped with a loading duct 301 and a grinder 302 of biomass 15. For example, the hopper 30 has a capacity of 8 cubic meters. A weighing system allows to measure the amount of added biomass 19. The feeding of biomass 19 to the pre-load tank 24 can be a periodic operation or a continuous operation. For a continuous feeding, it is necessary, for example, the inclusion of a storage cart with a worm-conveyor system, which feeds the pre-load tank 24 in a programmed manner.

Biomass 19 is mixed and homogenised with the fresh sewage 10 in the pre-load tank 24, which thus has a function of preparing a homogenised sewage 12. The homogenised sewage 12 is intended to feed the digester 4, i.e. it is a feeding sewage.

The pre-load tank 24 is connected to the pre-heating tank 26 through a pipe 34a, provided with a shut-off valve 34b and any pump (not shown), to transfer the homogenised sewage 12.

The pre-heating tank 26 has for example a capacity of 8 cubic meters and is provided with a heater, for example a coil pipe 36 in which a hot liquid is circulated. The homogenised sewage 12 is heated in the pre-heating tank 26 before being transferred to the digester 4. Optionally, the pre-heating tank 26 is equipped with a mixer (not shown) to improve heat exchange between the heater 36 and the homogenised sewage 12, thus speeding up the heating.

The pre-heating tank 26 has an outlet pipe 37a, equipped with a valve 37b, which connects it to the feed pump 28.

In the example, also the pre-load tank 24 is provided with an unload pipe 35a, provided with a bypass valve 35b, which connects it to the feed pump 28 without passing through the pre-heating tank 26, that is, by-passing the latter.

The feed pump 28 transfers the homogenised sewage 12 to the digester 4 through a feed pipe 38a provided with a shut-off valve 38b. The homogenised sewage 12 goes to mix with a sewage 14 which is already in digestion in the digester 4.

In a variant embodiment, one single tank provided with mixer 33 and heater 36 is used in place of the pre-load tank 24 and the pre-heating tank 26; the single tank therefore performs both the functions.

The tanks 21, 22, 24, 26 may be made of steel, metal, reinforced concrete or other material, according to known methods.

The digester 4 includes a digestion chamber 40, in which the sewage 14 in digestion is digested by the existing bacterial flora.

The digestion takes place under substantially anaerobic conditions by mesophilic bacteria, with production of biogas 18. In particular, the biogas 18 is a mixture containing mainly methane and carbon dioxide, with minor amounts of other components such as hydrogen, hydrogen sulphide and water.

The digester 4 includes a casing 5 of flexible material, which defines the digestion chamber 40. Basically, the casing 5, or bag or balloon, has a closed shape which encloses and bounds an internal zone corresponding to the digestion chamber 40.

The walls of the bag-like casing 5 are made with a layer or film of flexible material, which for example is a nylon fabric laminated to two layers of PVC. Furthermore, the walls of the casing 5 are fluid-tight; this can be achieved by using a flexible material which is already fluid-tight, or by means of sealing of a flexible material.

The casing 5 is suitable for containing the sewage in digestion 14, as well as for retaining the biogas 18 which is produced by the digestion.

The thickness of the layer of material is chosen so that the casing 5 has a sufficient strength to withstand the pressure inside the digestion chamber 40, i.e. the pressure of the biogas 18; for example, the thickness S5 of the layer of material of the casing 5 is of 4 mm.

In the example, the casing 5 (and therefore the digestion chamber 40) may have a volume from 1000 to 5000 cubic meters. By way of illustration, the casing 5 has a length L5 of 45 m, a width P5 of 25 m and a height H5 of 3 m.

Basically, the casing 5 has a substantially horizontal development, wherein the casing 5 has a height H5 that is less than the length L5 and the width P5 of the casing 5 itself.

As shown in the figures, the casing 5 has a top wall or face 51, a bottom wall or face 52 and a lateral wall or face 53 that connects the top face 51 and the bottom face 52 to each other. In other words, the lateral face 53 is a peripheral face of the casing 5 and is between the top face 51 and the bottom face 52.

In the example, the bottom face 52 has a surface extension which is less than the surface extension of the top face 51; the lateral face 53 is therefore tilted relative to the top face 51 and the bottom face 52. Thus, the casing 5 substantially has a shape of a truncated pyramid turned upside down.

In any case, it is evident that the casing 5 can have different shapes, such as a parallelepiped or a disc.

It should be noted that the construction of the bag-like digester 4 is particularly simple and inexpensive in comparison to the plants of the known art. In fact, the construction of the digester 4 does not require any complex building works and does not use expensive materials such as concrete and steel.

The bag-like casing 5 is supplied as a prefabricated member with the desired size and volume, designed according to the capacity required for the plant 1. The bag 5 is supplied in a loose condition, so as to be easily transported to the construction site of the digester 4.

The plant 1 comprises a compartment or basin 91 configured to at least partially receive the casing 5. The bottom face 52 of the casing 5 rests on a first surface 91a of the compartment 91.

Basically, the bottom face 52 is supported for its entire extension by the first surface 91a, being positioned substantially fitting with the first surface 91a. This is made possible also by the flexibility of the material of the casing 5.

In the embodiment shown, the casing 5 is at least partially underground, i.e., the compartment 91 is a trench or pit made into a ground 90 and having a depth H9 of one or two meters relative to the level 95 of the ground 90.

A raised containment bead 96 surrounds the perimeter of the casing 5 projecting from the pit 91; the containment bead 96 is made using the same material from the excavation, without any filling material.

Basically, the bottom face 52 of the bag-like casing 5 is at a level that is lower than the ground level, whereas the top face 51 is at the level of the bead 96 or at a higher level.

In the embodiment shown in the figures, the top face 51 is at a level higher than the level 95 of the bead 96, i.e. the height H5 of the casing 5 is greater than the depth H9 of the pit 91.

When the casing 5 is installed in the pit 91, the bottom face 52 of the casing rests on the bottom surface 91a of the pit 91, which is therefore a first supporting surface. Similarly, the underground portion of the lateral face 53 rests on the lateral surface 91b of the pit 91, which is therefore a second supporting surface on which the lateral face 53 is at least partially supported.

The bottom face 52 and/or the lateral face 53 rest on the pit 91 via their outer surface 52a, 53a, that is, via the surface which is on the opposite side relative to the digestion chamber 40 or internal zone of the casing 5.

In particular, the entire extension of the bottom face 52 and at least a part of the lateral face 53 are supported by the ground 90, these faces 52, 53 being positioned substantially fitting with the surfaces 91a, 91b of the pit 91, i.e. with the supporting surfaces for the casing 5.

The bag-like casing 5, once inserted into the pit 91, is constrained to the ground 90 by means of first tie members 58a that connect the top face 51 of the casing 5 to respective first stakes 59a driven into the ground or in the containment bead 96. In the example, the first tie members 58a and the first stakes 59a are arranged along the entire perimeter of the casing 5, so as to ensure a stable positioning of the casing 5 and a sufficient stretching of the top face 51, to keep it taut.

Second tie members 58b and second stakes 59b that connect the bottom face 52 of the casing 5 to the bottom 91a of the pit 91 are provided as well.

For example, the tie members 58a, 58b are flexible cables or ropes.

In order to reduce heat loss, a cloth 48 of heat-insulating material (for example, a flexible sheet of fibreglass) is positioned between the bag-like casing 5 and the bottom and lateral surfaces 91a, 91b of the pit 91, so that the heat exchange between the sewage in digestion 14 and the ground 90 is minimized.

The plant 1 further comprises a heater for heating the sewage in digestion 14 which is inside the digestion chamber 40.

In one embodiment, a heater of the casing 5 is arranged in the pit 91. In the example, the heater is a coil pipe 45 which is positioned between the casing 5 and the surfaces 91a, 91b of the pit 91; in particular, the coil pipe 45 is peripherally arranged around the lateral face 53 of the casing 5, i.e. it is positioned between this lateral face 53 and the lateral surface 91b of the pit 91. Alternatively, the coil pipe 45 may be arranged at the bottom face 52 of the casing 5, i.e. positioned between this bottom face 52 and the bottom surface 91a of the pit 91, or may be arranged at both the bottom face 52 and the lateral face 53.

Alternatively (as discussed later), the coil pipe 45 is arranged inside the bag-like casing 5, i.e. directly in the digestion chamber 40.

As it will be clearer in the following, a hot fluid is circulated in the coil pipe 45 and, thanks to the contact between the coil pipe 45 and the casing 5, a heat transfer from the circulating hot fluid to the digestion chamber 40 takes place.

In particular, the coil pipe 45 is positioned between the casing 5 and the cloth 48 of heat-insulating material.

Even in the case where the coil pipe 45 and/or the cloth 48 are positioned between the casing 5 and the ground 90, it can be said that the resting of the bottom face 52 and/or of the lateral face 53 of the casing 5 on the supporting surface 91a, 91b is “fitting”; in fact, apart from the depressions and the additional thickness due to the coil pipe 45, the faces 52, 53 of the casing 5 are flexible and conforming to the shape of the respective supporting surface 91a, 91b on the ground 90, following and copying the trend thereof.

In the example, the feed pipe 38a of the feeding sewage 12 and the unload pipe 61 of the digestate 16 are buried below the casing 5.

The feed pipe 38a is communicating with the inside of the casing 5, i.e. with the digestion chamber 40, in one or more inlet points. In the example, the feed pipe 38a has a main tube 38p, from which a plurality of branches 38d depart; each branch 38d passes through the bottom wall 52 of the casing 5 and ends in the digestion chamber 40, where it has a mushroom-shaped inlet end 38t.

One or more outlet points, through which the digested sewage (or digestate 16) is removed from the casing 5, are provided as well. In the example, the bottom wall 52 of the casing 5 has an opening or pit 60 which is connected to an unload pipe 61.

The unload pipe 61 can be provided with a shut-off valve 61b and/or a pump 61c.

As shown in the figures, the inlet points 38t and the outlet points 60 for the sewage are distant from each other, for example on opposite sides along the length L5, so that the feeding sewage 12 has a sufficient residence time in the digestion chamber 40.

Inside the casing 5, the digester 4 is further provided with one or more mixers or stirrers 55 for the sewage in digestion 14, to keep the homogeneity of the latter in the digestion chamber 40 as much as possible. In particular, the stirrers 55 are of the submerged type.

The operation of the stirrers 55 is not continuous, but it is a function of the temperature of the sewage bulk 14.

The casing 5 comprises one or more ports 57 of extraction of produced biogas 18, which for the example are openings made in the top wall 51 of the casing 5; the extraction ports 57 are connected via flexible tubing 71 to the feed pipe 72 of the gas engine 81.

An overpressure and low-pressure safety valve is installed on the feed pipe 72, in order to ensure the operational safety of the casing 5.

The casing 5 may also comprise one or more safety valves 49, for example arranged in the top wall 51, which are calibrated to automatically discharge the biogas 18 from the digestion chamber 40 when the pressure inside the latter exceeds a predetermined threshold value.

Passages 56 (or manhole hatches) for going into the internal chamber 40, for example for installation and/or maintenance reasons, are provided on the casing 5. The hatches 56 have a hermetically-sealed closing.

The digester 4 includes an apparatus 46 for reduction of the sulphur content in the biogas. In the example, the reduction apparatus 46 operates by injecting oxygen in the casing 5 and is connected to the digestion chamber 40 through a pipe 46a which ends in the digestion chamber 40, in which it enters through an opening in the top wall 51. The oxygen is metered through a control unit which continuously takes the percentage of hydrogen sulphide in the biogas.

The produced biogas is used as a fuel in a co-generator 8 that simultaneously produces electric energy and heat. The heat produced from the cooling water and from the exhaust gases of the engine 81 during its operation is recovered through heat exchangers and used for heating the digester 4.

As indicated in the figures, the hot water produced by the recovery exchangers is sent to the heating coil of the digester 4 via a piping and a circulation pump.

In particular, the sewage 14 is heated to a temperature above 35° C. and at pH 7.5, so as to allow the mesophilic bacteria to work in optimal conditions and to produce biogas.

In the case where the digestion process requires a heat quantity greater than that provided by the heat recovery from the engine 81, a boiler arranged in the recovery circuit, in parallel or in series to the recovery exchangers of the engine 81, may be provided.

Similarly, a boiler may be provided in the case where the co-generator 8 is not present and then the heating of the digester 4 should be made with a special heating circuit.

A temperature sensor 140 may be provided to measure the temperature of the sewage 14 inside the digestion chamber 40 and to accordingly adjust the heating system of the digester 4.

The plant 1 is provided with a central unit, or control board, by which the working of the plant 1 is monitored and the operative conditions can be varied, such as opening/closing of valves, switching on/off of pumps, stirrers and sulphur reduction apparatus 46, adjustment of the operating condition of the co-generator 8 or the boiler.

With particular reference to the embodiments shown in the figures, more details are provided below.

The section 6 of digestate treatment comprises a tank 63 into which the unload pipe 61 conveys the digestate 16 removed from the casing 5.

The digestate tank 63 is for example a decanter-separator, which separates the digestate 16 into a heavier fraction (for example, more solid-rich) and a lighter fraction. A first part of the digestate (for example, the heavier fraction) is removed from the plant 1 through respective piping 64 and sent to subsequent treatments (for example drying and/or re-using in the agricultural field); a second part of the digestate (for example, the lighter fraction) is recirculated and fed back to digestion through recirculation piping 65.

In the embodiment shown in FIG. 1, the recirculation piping 65 is configured to inlet the recirculated digestate into the pre-heating tank 26, in which the recirculated digestate is mixed with the fresh sewage 10 to obtain the homogenised sewage 12.

A valve 65b on the recirculation piping 65 allows to adjust the inlet of the recirculated digestate into the pre-heating tank 26.

The pipes 61, 64, 65 for the digestate can be provided with appropriate valves and pumps (not shown).

In an alternative embodiment, the recirculated digestate is sent directly into the digestion chamber 40 without passing through the pre-heating tank 26: for example, the recirculation piping 65 is connected to the feed pipe 38a.

The tubes 71 for extracting the biogas 18 are connected to a biogas conditioning section 7, from which the biogas 18 is sent to the co-generator 8 (or alternatively to a storage tank) through a respective pipe 72.

The conditioning section 7, for example, performs the pumping, filtration and analysis of the biogas 18. Where appropriate, the conditioning section 7 can perform a dehumidification of the biogas 18.

In one embodiment, the conditioning section 7 and the co-generator 8 are installed in a single module (for example, in a container with dimensions of 12 m×2.5 m×3 m) provided with a control unit.

The tubes 71, 72 basically compose a biogas line and are parts of fuelling means to fuel the co-generator 8 with the biogas 18 produced by the plant 1.

The biogas 18 is used as a fuel by the engine 81 of the co-generator 8, to produce electric energy and heat.

In the circuit 83 for cooling the engine 81, water (or other suitable liquid) is circulated and cools down the engine 81 by removing from it the heat produced by combustion of the biogas 18.

The heat thus recovered is used for heating the digester 4 and/or the pre-heating tank 26.

As shown in FIG. 1, the cooling circuit 83 is connected to a pipe 42 which has a first branch 42a feeding the coil pipe 45 of the heater of the casing 5, and a second branch 42b feeding the coil pipe 36 of the heater of the pre-heating tank 26.

The water coming out from the coils 45, 36 is fed, via respective outlet pipes 43a, 43b, to a return pipe 43 which is connected to the cooling circuit 83 of the engine 81. The cooling circuit 83, the pipes 42, 42a, 42b, 43, 43a, 43b and the coils 36, 45 compose a circuit 87 of recovery and reuse of heat.

In the circuit 87 of recovery and reuse of heat, a shut-off/control valve 44 and a pump (not shown) for the circulation of the cooling/heating water are provided.

Basically, the biogas production plant 1 and the co-generator 8 are connected to each other by connecting piping, which includes the pipes 42, 43 of the circuit 87 of recovery and reuse of heat and the tubes 71, 72 for feeding the biogas 18.

In a second embodiment, shown in FIG. 2, a heat exchanger 68 between the recirculated digestate and the circuit 87 of recovery and reuse of heat is provided. The heat exchanger 68 is positioned outside the bag-like casing 5.

In particular, a third branch 42d of the pipe 42 feeds hot water to the heat exchanger 68, which is arranged on the recirculation piping 65.

The water coming out from the exchanger 68 is fed to a return piping 43 through an outlet pipe 43d.

The recirculated digestate, after being heated in the exchanger 68, is introduced into the casing 5 without going through the pre-heating tank 26. For example, the recirculation piping 65 introduces the recirculated digestate directly into the feed pipe 38a. The recirculation piping 65 is provided with a valve 65b and a pump 66.

As shown in FIG. 10, a regulation system, comprising a temperature sensor 681 and a control valve 682 (for example a three-way valve) controlled by a control unit of the regulation system, can be provided. The temperature sensor 681 measures the temperature of the heated digestate going out from the exchanger 68; the control unit, by means of the control valve 682, regulates the flow of hot water going into the exchanger 68 so as to obtain a desired temperature for the digestate going out from the exchanger 68. The control valve 682, in practice, regulates the flow of hot water which is diverted to the branch 42d of the pipe 42.

In particular, the exchanger 68 is a concentric-pipe exchanger (FIGS. 11 and 12), i.e. having an outer tube in which one of the two fluids (e.g., the hot water) flows, and an inner tube which is inside the outer tube and in which the other of the two fluids (e.g., the digestate to be heated) flows.

In a different embodiment, the heating of the sewage 14 contained in the digestion chamber 40 is entirely or mainly obtained through the exchanger 68. In other words, the heating of the sewage 14 is obtained by means of an external exchanger 68 which heats up an amount of sewage drawn out of the bag-like casing 5; the heated sewage is introduced again into the bag-like casing 5. In other words, the heating is carried out on a recirculation line or branch for the sewage 14.

In particular, the heating fluid (i.e., hot water) is obtained from the circuit 87 of recovery and reuse of the heat from the engine 81.

In this different embodiment, the coil pipe 45 around the outside of the bottom face 52 of the bag-like casing 5 or inside the bag-like casing 5 may not be present, because the heating of the sewage in digestion 14 can be fully obtained by directly heating the recirculated sewage in the exchanger 68.

In particular, the outlet pit 60, from which the sewage to be recirculated is drawn out, is arranged on the bottom of the bag-like casing 5.

In the embodiments shown in FIGS. 13 to 18, the compartment 91 has a countersunk shape having walls tilted towards a bottom region. Therefore, the bottom face 52 of the bag-like casing 5, which rests on the compartment 91, has a concave shape, in which a central region 52c is at a minimum level. The outlet pit 60 is located at said central region 52c at the minimum level. This is useful for drawing out the sewage 14 from where this is cooler and denser, so as to maximize the effect of heating and recirculation of the biomass comprised in the sewage.

The unload pipe 61 connected to the outlet pit 60 feeds, through a pump 66, the exchanger 68.

A recirculation/feed pipe 65 connects the sewage-side output of the exchanger 68 to a main feed tube 38p, which for example is ring-shaped and is arranged around the perimeter of the bag-like casing 5, substantially at the level 95 of the ground 90 or underground.

For convenience of representation, in the schematic representations of FIGS. 13, 14 and 33, the main ring tube 38p is shown both just above ground (where only its sectional views are shown) and above the bag.

Branches 38d depart from the main tube 38p, the branches 38d introducing (heated) sewage into the bag-like casing 5 substantially at the free surface of the sewage 14 in the digestion chamber 40. The branches 38d go through the lateral wall 53 of the bag-like casing 5 at openings that are hermetically sealed, for example according to a mode described below.

In particular, the branches 38d include a flexible portion 380 positioned between the main tube 38p and the lateral wall 53 of the bag-like casing 5, so as to follow the deformations of the bag-like casing 5 during the loading/unloading of the same and its shape changes in general.

In the embodiments illustrated in FIGS. 15 to 18, the branches 38d go into the bag-like casing 5 and are directed so as to introduce the sewage with a direction substantially parallel or tangential to a respective flank of the bag-like casing 5. In particular, the inlet directions of the branches 38d are concordant with each other: for example, in a plan view, all the branches 38d are configured to introduce sewage according to a clockwise direction, or all are configured to introduce sewage according to a counter-clockwise direction.

In the embodiment shown in FIGS. 15 and 16, the bag-like casing 5 has a square shape in a plan view and has a volume of 1,500 cubic meters, for example. There are four inlet branches 38d, each at a respective vertex of the square and oriented in a clockwise direction along a respective side of the square. Moreover, only one outlet pit 60, which is arranged at the bottom substantially at the centre of the square, is provided.

In the embodiment shown in FIGS. 17 and 18, the bag-like casing 5 has a rectangular shape in a plan view and has a volume of 3,000 cubic meters, for example. There are four inlet branches 38d, each at a respective vertex of the rectangle and oriented in a clockwise direction along a respective side of the rectangle. Moreover, two outlet pits 60 arranged at the bottom, spaced from each other and aligned along an axis of symmetry of the rectangle, are provided.

In other words, a sewage recirculation line comprises an outlet piping 61 that draws sewage 14 out of the digestion chamber 40 and an inlet piping (for example composed by the main tube 38p and the branching 38d) that re-injects the sewage into the digestion chamber 40. The heat exchanger 68 is positioned between the outlet piping and the inlet piping, i.e., it heats up the drawn-out sewage before the latter is sent back into the bag 5.

The injection of recirculated sewage at the surface, i.e. near the free surface, is advantageous for a continuous mixing of the sewage 14. In fact, the sewage is drawn out from the bottom of the bag-like casing 5, where it thickens more, and is injected at the free surface of the liquid; thanks to this, the biomass is brought back to the surface, thus avoiding stagnation at the bottom.

In addition, the injection along the flanks of the bag-like casing 5 ensures a flow tangential to the flanks themselves and cleaning the flanks, by stirring stagnations on the flanks and at the corners. The lateral faces of the bag-like casing 5 are tilted towards the centre of the bottom and then, in addition to the tangential flow, a flow by gravity is established over the whole surface of the lateral faces as well.

The concordant inlet directions are useful for obtaining a surface recirculation (clockwise or counter-clockwise), which improves both the mixing of the sewage 14 and the cleaning of the flanks of the bag-like casing 5.

The mixing carried out by the sewage recirculation is then in addition to the action of the stirrers 55 (not shown in FIGS. 13 to 18).

As shown in FIGS. 13 and 14, the main tube 38p and the branches 38d can also be used for loading fresh sewage into the bag-like casing 5.

A load pipe 37a gets into the sewage recirculation piping, for example upstream of the pump 66 and of the heat exchanger 68.

A proper valve system, such as a three-way valve 370, is provided to alternately manage the sewage loading (during a starting step or a sewage replenishing step, FIG. 13) and the sewage recirculation (during a step of continuous working of the plant, FIG. 14). For example, during the loading of the sewage, the unload pipe 61 is closed and the load pipe 37a injects sewage into the bag-like casing 5 through the main tube 38p and the branches 38d; during the operative working of the digester 4, the load pipe 37a is closed and the unload pipe 61 draws sewage out of the bag 5 to recirculate, heat up and inject the sewage back into the bag-like casing 5 through the same main tube 38p and branches 38d.

As shown schematically in the figures (in particular in the sectional view of FIG. 5), the construction of the digester 4 first of all provides to make an excavation into the ground 90 to obtain a pit 91 having a shape complementary to the portion of the bag 5 to be positioned underground. In the pit 91, the feed pipe 38a and the unload pipe 61 for the digester 4 are suitably positioned.

In particular, before positioning the bag-like casing 5, the following operations are carried out inside the pit 91:
a bed 92b of middle-sized gravel of about 20 cm is laid at the bottom of the pit 91;
a pipe 93 for draining rainwater (which may seep from the edges of the pit) is positioned and buried in the bed of gravel 92b; the drain pipe 93 is connected to a manhole 94a provided with a submerged pump 94b;
the pit 91 is covered by a steel mesh adapted to protect the bag 5 from assaults by rodents or coypus; in particular, the steel mesh is laid under the bed of gravel 92b;

Furthermore, the following items are included in the pit 91:
a pipe 38a of PVC pre-insulated with polyurethane, for feeding the sewage;
a pre-insulated pipe 61 of PVC, for drawing out the sewage;
the anchoring plinths 501 of the heating coil 45 (if placed directly inside the bag);
the anchoring plinths 501 of the stirrers 55 of the sewage;

The pit 91 is then filled, up to the level of the anchoring plinths 501, with fine rounded gravel and soil, to form a bed 92a. Overall, the gravel beds 92a and 92b compose a draining bottom 92.

In case, the heat-insulating cloth 48 is laid in the pit 91.

If the coil pipe 45 is provided outside the bag-like casing 5, the coil pipe 45 is placed on the cloth 48 or on the draining bottom 92. It should be noted that FIG. 5 shows a sectional view of the coil pipe 45; however it is clear that, for example, the coil pipe 45 is comprised of a single tube wound up to form a plurality of turns.

If the coil pipe 45 is instead provided inside the bag 5, it is preferable that the feed pipe 38a comes up in the bag 5 substantially at the centre of the latter, so that the sewage loading takes place in the centre of the bag and then in the centre of the coil, where the heat of the heating has the highest concentration.

If the coil pipe is not provided for at all, because only a heating via an external exchanger 68 is used, the relevant steps of preparation and assembly are not required. Similarly, if the feed pipe 38a is of the ring type 38p with surface inlets 38d instead of being buried, the steps above are modified accordingly.

At the end of these operations, the casing or bag 5 is placed into the pit 91 and the mounting, inside it, of the equipments necessary for its working as a digester 4 starts.

By means of a lifting crane, the upper part 51 of the bag 5 is lifted to allow:
fastening of the upper part 51 of the bag 5 to the bead 96 through the pickets or stakes 59a and the sprung tie members 58a;
entering inside the bag 5, through the existing passages 56, and fastening on the appropriate previously-arranged plinths the electric stirrers 55 for the sewage and the support brackets and stainless steel pipes that compose the coil 45 for heating the sewage (if the heater is provided inside the bag 5).

Moreover, the feed pipe 38a of the sewage 12 and the unload pipe 61 of the digestate are connected to the bag 5 and the junctions between pipes 38a, 38d, 61 and the bag 5 are sealed in an appropriate manner so that there are no leaks.

On the top of the bag 5, the extraction ports 57 for the biogas (which are connected to the tubes 71 for extracting biogas; the junctions between the tubes 71 and the bag 5 are sealed in an appropriate manner so that there are no leaks) are fastened and the passages 56 are hermetically closed.

Basically, the bag 5 is closed so as to separate an internal zone of the bag 5 from an outer zone. The bag 5 is so adapted for containing the sewage 14 and defines the digestion chamber 40. The bag 5 is ready to be loaded with sewage.

The plant 1 may also include a storage bag or second digestion bag, which will be described in more detail below.

Some examples of operational modes for the working of the plant are described below.

### Mode 1—Using Only Pig Sewage

The sewage goes into a sedimentation tank to raise the concentration by weight of the available organic substance (7-8%).

The sewage is pumped from the sedimentation tank, by means of a discontinuously-operating pump, into the digestion bag, whereas the excess liquid in the sedimentation tank is sent by gravity to a storage bag of the plant.

The discontinuous feeding of the plant allows to equalize the characteristics of the sewage inside the digestion bag, thus avoiding any ‘shock’ to the bacterial flora due to a sudden lowering of the digestion temperature when fresh sewage is introduced. To overcome this drawback, in the case of continuous feeding of the plant, a shell-and-tube heat exchanger is included in the feed pipe; the shell-and-tube heat exchanger pre-heats the inlet sewage using the heat of the outlet sewage.

After one day of residence of the fresh sewage in the digestion bag, an amount of digestate equal to the feeding amount is removed from the digester via the outlet pipe and the pump, and it is sent to the storage bag where its digestion is completed.

This type of digestion allows a production of 20-25 cubic meters of biogas per tonne of treated sewage.

### Mode 2—Use of Cattle Sewage

When necessary, the sewage and excrements in the collection tanks of the stables should be diluted to an organic substance concentration of 10-12% by weight, otherwise they could not be pumped.

The sewage is pumped from the collection tank, by means of a discontinuously-operating pump, into the digestion bag.

The discontinuous feeding of the plant allows to equalize the characteristics of the sewage inside the digestion bag, thus avoiding any ‘shock’ to the bacterial flora due to a sudden lowering of the digestion temperature when fresh sewage is introduced. To overcome this drawback, in the case of continuous feeding of the plant, a shell-and-tube heat exchanger is included in the feed pipe; the shell-and-tube heat exchanger pre-heats the inlet sewage using the heat of the outlet sewage.

After one day of residence of the fresh sewage in the digestion bag, an amount of digestate equal to the feeding amount is removed from the digester via the outlet pipe and the pump, and it is sent to the storage bag where its digestion is completed.

This type of digestion allows a production of 30-40 cubic meters of biogas per tonne of treated sewage.

Mode 3—Use of Pig or Cattle Sewage with Added Vegetal Silage

The digester is fed with a substrate consisting of a mixture of solid biomass (which usually is corn silage or another silage) and sewage, which are mixed in a pumping and mixing station.

A hopper with an automatic weighing and unloading system for the silage feeds a pumping station, where fresh sewage is introduced at the same time.

The organic substance concentration may exceed 12% by weight.

The sewage is discontinuously pumped into the digestion bag, thus avoiding any ‘shock’ of the bacterial flora due to a sudden lowering of the digestion temperature.

To overcome this drawback, in the case of continuous feeding of the plant, a shell-and-tube heat exchanger is included in the feed pipe; the shell-and-tube heat exchanger pre-heats the inlet sewage using the heat of the outlet sewage.

After one day of residence of the fresh sewage in the digestion bag, an amount of digestate equal to the feeding amount is removed from the digester via the outlet pipe and the pump, and it is sent to the storage bag where its digestion is completed.

This type of digestion allows a production of 150-200 cubic meters of biogas per tonne of treated sewage.

### Mode 4

The working of the plant 1 is described in greater detail below, with particular reference to the embodiments shown in the figures.

The sewage 10 collected from the farm is progressively stored in the first storage tank 21 or in the second storage tank 22, depending on which of the two has a sufficient free volume.

The feeding of sewage to the digester 4 is discontinuously carried out. For example, four cubic meters of homogenised sewage 12 are fed to the digester 4 every two hours in a single loading operation and a corresponding amount of digestate 16 is removed from the digester 4 and sent to a tank or a storage bag. In the example, the sewage 12 fed to the digester 4 via the feed pipe 38a (and therefore contained in this pipe 38a) has an organic substance concentration that is less than 15% by weight, given by the mixing between the fresh sewage 10 and the recirculated digestate.

During the feeding operation, the bypass valve 35b is closed, the valves 37b and 38b are open and the pump 28 is operating. A predetermined amount of homogenised and preheated sewage 12 is transferred from the pre-heating tank 26 to the digestion chamber 40 via the feed pipe 38a. Once the operation is completed, the pump 28 is stopped and the valves 37b, 28b are closed.

A corresponding amount of homogenised sewage is transferred from the pre-load tank 24 to the pre-heating tank 26 via the pipe 34a. The sewage in the pre-load tank 24 is replenished by a draw from the first storage tank 21 or from the second storage tank 22. If necessary, the hopper 30 is opened to add a fixed amount of biomass 19 into the pre-load tank 24.

Therefore, in the time which elapses until the next feeding operation of the digester 4, the sewage (and the biomass 19, in case) in the pre-load tank 24 is mixed and homogenised by means of the mixer 33. The sewage 12 in the pre-heating tank is heated, thanks to the heater 36, up to a desired temperature.

It should be noted that each feeding operation of the digester 4 involves only about half the volume of the pre-load tank 24 and the pre-heating tank 26. In this way, the fresh sewage 10 subsequently introduced into these tanks 24, 26 is mixed with an equal amount of already homogenised sewage 12, which is like the one already fed to the digester 4. This allows to equalize the characteristics of the sewage 12 fed to the digester 4, preventing it from having very different characteristics from a feeding to the next feeding; any “shock” of the bacterial flora, which may be caused by feedings too different from each other, is avoided.

In the event that pre-heating of the sewage 12 is not necessary, the feeding to the digester 4 can be made directly from the pre-load tank, by opening the bypass valve 35b instead of the feed valve 37b; the bypass valve 35b is then closed after this operation.

Periodically, for example with the same time rate of the feeding of sewage 12, a certain amount of digestate 16 is removed from the digester 4 via the unload pipe 61. After passing in the digestate tank 63, a first part of the digestate 16 is removed from the plant 1, while a second part (or recirculation part) is transferred to the pre-heating tank 26. The recirculated digestate is then mixed and heated together with the fresh sewage. Thanks to this, the part of the bacterial flora that is contained in the digestate 16 is reintroduced into the digester 4, thus avoiding the bacterial depletion of the latter.

Alternatively, the recirculated digestate is fed directly to the digester 4, if necessary after a heating in the exchanger 68.

For all operational modes mentioned above, the anaerobic digestion of sewage takes place in the digestion chamber 40, i.e. inside the bag 5, to produce biogas 18. The digestion takes place continuously. The produced biogas 18 moves upward and gathers in the upper part of the bag 5, above the free surface of the sewage in digestion 14.

In the digestion chamber 40, the sewage is kept at a temperature of about 35° C. and at a pH of about 7.5.

During digestion, the sewage 14 is intermittently mixed by the mixers or stirrers 55, in order to keep it as homogeneous as possible. When necessary, the apparatus 46 for reduction of the sulphur content is activated to inject oxygen into the bag 5.

In addition, iron hydroxide can be added to the sewage, in order to reduce the production of hydrogen sulphide during digestion. For example, iron hydroxide is added to the recirculated sewage via a metering pump 460.

The sewage in digestion 14 inside the bag 5 is a mixture of fresh sewage 12 and partially-digested sewage. In the interval between one feeding operation and the other, the digestion proceeds with depletion of the organic matter, which is then restored at each feeding. It should be noted, however, that the volume of the bag 5 and then of the sewage in digestion 14 (for example, 3000 cubic meters) is quite greater than the amount fed during each operation (for example, 4 cubic meters).

Since the residence times of sewage in the digester are very long, the overall composition changes very little during each feeding operation.

In particular, during the working of the plant 1 the level of the sewage in digestion 14, that is the level of the liquid phase in the bag 5, is maintained at a height lower than the level 95 of the bead 96. In other words, the height H14 of the free surface of the sewage 14 in the digestion chamber 40 is lower than the depth H9 of the containment pit 91.

In this way, the weight of the sewage 14 is entirely discharged on the bottom surface 91a and the lateral surfaces 91b of the pit 91, avoiding the risk that the weight may compromise the stability of the bag 5 on the ground 90.

In the embodiment described above, the height H14 of the sewage 14 is 3 m at the most and then a height H18 of at least 0.5 m is available for the biogas.

The produced biogas 18 is continuously removed through the extraction tube 71. The extraction is carried out while trying to keep constant the pressure inside the bag 5, which for example is between 2 and 15 mbar.

In the case where the internal pressure excessively rises, for example because of a stopping of the biogas extraction or a shutdown of the engine 81, the pressure-relief safety valve 49 opens and discharges the biogas 18 to the outside, so preventing the breaking of the bag 5.

The biogas going out from the digestion bag is sent, via a tube of PVC, to a gasometer for stabilizing the pressure.

The biogas extracted from the gasometer goes through a cooling exchanger where it loses moisture of which it is saturated; the engine of the co-generator is fuelled with biogas by means of a blower.

In other words, the biogas 18 is sent to the co-generator 8, after conditioning in the conditioning section 7. The engine 81 uses the biogas 18 as a fuel and drives the alternator 85, with production of electric energy which is transferred to an accumulator or to an electric network 89.

Since the biogas 18 is continuously produced and extracted, also the co-generator 8 runs continuously.

The heat produced by combustion of the biogas 18 in the engine 81 and the residual thermal energy of the flue gases are transferred to the water circulating continuously in the recovery circuit 87.

The hot water thus obtained, at the outlet of the cooling circuit 83 of the engine 81, circulates in the coil 45 and/or in the exchanger 68. A heat transfer to the digestion chamber 40, which is maintained at an optimal temperature for digestion, takes place.

A part of the hot water heats the homogenised sewage 12 in the pre-heating tank 26 (by means of the coil 36), preparing it for the next feeding to the digester 4, and/or the recirculated digestate in the heat exchanger 68.

The cooled water, going out from the heat exchangers 36, 45, 68, goes back to the cooling circuit 83 of the engine 81, where it is heated again. The water is then continuously circulated in the recovery circuit 87: during the working of the plant the valve 44 is open and the circulation pump (not shown) is on.

When the animals of the farm are subjected to an antibiotic treatment, there is the risk that the fresh sewage 10 is contaminated by the antibiotic and that it could damage the bacterial flora, thus compromising the digestion and the working of the plant 1.

In this case, it is possible to send the sewage directly to a storage bag.

Basically, a plant 1 according to the present disclosure allows to have a digestion chamber 40 having a very large volume at a low cost.

Thanks to this, it is possible to have average residence times for the sewage 14 in the digester 4 which are very high, even in the case of a significant amount (20-50 cubic meters per day) of produced sewage to be treated. The plant 1 can therefore operate under conditions of temperature and organic substance concentration in the sewage which do not correspond to the maximum activity of the bacterial flora. Basically, compared to prior art plants, the lower digestive activity is balanced by a much longer time available for digestion.

The low cost of the plant 1, thanks to its simple construction, makes acceptable even a yield of the plant 1 (calculated as daily production of biogas per unit volume of the digester) which is much lower than the known plants.

Consequently, the addition of “valuable” biomass to the sewage is required only in emergency cases (as mentioned above), not as a standard practice, and it is also possible to fully exploit the organic load of “poor” sewage (i.e., with reduced concentration of organic substance), which otherwise could not be used and should be disposed of even bearing the consequent costs.

In another embodiment of the plant 1, shown in FIG. 19, the heater of the digestion chamber 40 is positioned inside the digestion chamber 40 itself. This is useful for having a high-efficiency heating, thanks to the fact that the sewage 14 in the bag 5 is in direct contact with the heater.

The heater comprises a coil pipe 45 which is mounted in the bag 5, and a connection piping which connects the coil pipe 45 to the circuit 87 of recovery and reuse of heat.

The connection piping comprises an inlet or feed pipe 42a and an outlet or return pipe 43a. In the following, reference will be made only to the feed connection pipe 42a; it is however clear that the same features can be applied also to the return connection pipe 43a.

The coil pipe 45 is composed by pipe lengths 451 hydraulically connected to each other and is supported by upright supports 453 mounted inside the bag 5; in the example, each upright support 453 includes brackets 455 for supporting the respective pipe lengths 451.

The connection pipe 42a, in its portion within the digestion chamber 40, comprises at least one flexible portion 421. Thanks to the flexible portion 421, the portions 420a, 420b of the connection pipe 42a that are upstream and downstream of the flexible portion 421 (joined to it by means of flanges) can move relative to each other.

This is useful during the installation phase of the digester 4, when the coil pipe 45 in the bag 5 is to be connected to a length 420a that is already mounted in the ground 90: in fact, the capability of relative movement allows less strict tolerances for the design and the assembly of the coil pipe 45 and the upright supports 453. Furthermore, the flexible portion 421 allows the heater to cushion any deformation due to thermal expansion of the coil pipe 45.

The installation of the heater inside the bag 5 is carried out by working directly in the bag 5. The pipe lengths 451 are introduced into the bag 5 through the hatches 56; the installing workers 480, that go inside through the hatches 56, mount the upright supports 453 and the pipe lengths 451 to each other, to obtain the coil pipe 45. Basically, the heater is assembled inside the bag 5.

In order to prevent the loose bag 5 from hampering the assembling operations, removable arches 458 can be temporarily installed on the upright supports 453 for supporting the top wall 51, holding it raised from the ground, so as to leave a sufficient room for the workers 480 (FIG. 20). The removable arches 458 are removed after the installation of the heater and are brought out of the bag 5 through the hatches 56.

A sealed connection mode between the walls 52, 53 of the bag 5 and the pipes entering the bag 5 is shown in FIG. 21; in the example shown in FIG. 21, such pipes are a length 420a of the connection pipe 42a of the heating coil 45 and a length 610 of the unload pipe 61.

Before positioning the bag 5, the pipe lengths 420a, 610 are placed in the ground 90 and are partially protruding from it. Around each pipe length 420a, 610, a plinth or base 501 of concrete is casted, which is sunk into the ground 90 and at the level of the respective surface 91a, 91b. A first seal 503 (for example, made of silicone material) is placed around the pipe length 420a, 610 and resting on the plinth 501. After this, the bag 5, which is provided with openings in which the respective pipe lengths 420a, 610 are inserted, is positioned. A second seal 505 is placed around the pipe length 420a, 610 on the inner face of the respective wall 52, 53 of the bag 5; the second seal 505, which is made of thermoplastic or silicone material, is heat-sealed on the wall of the pipe length 420a, 610, so as to seal the wall 52, 53 to the pipe 420a, 610. In order to ensure the stability of the connection and the compression of the seals 503, 505, screw anchors or screws 507 are used and are screwed from the inner side of the bag 5 into the plinth 501, so as to cross the second seal 505, the wall 52, 53 of the bag 5 and the first seal 503.

Alternatively, instead of the second seal 505, a closure plate 506 can be used (see FIG. 31).

Similar modes are used to install the stirrers 55 and the upright supports 453 for supporting the coil pipe 45. Also in this case, a plinth 501 of concrete is prepared, on which a first seal 503, the bottom wall 52 of the bag 5, and the upright support 453 resting by its base 453a are positioned in order; the whole is fastened by screws 507. Optionally, a second seal 505 can be placed between the bottom wall 52 of the bag 5 and the base 453a of the upright support 453.

FIGS. 22, 23, 30 and 32 show a further embodiment of a plant 1 for the production of biogas.

As an example (that, of course, can be applied to other embodiments as well), a perimeter fence 99 and a stable 98 are also shown.

In addition to the digester 4, which is entirely analogous to that already described above, the plant 1 comprises a storage 400 for the digested sewage 16 leaving the digester 4 and waiting for a final disposal. Basically, the plant is composed, in addition to the digestion bag, also by another bag having the same constructional size and acting as a storage for the digested sewage. The second bag comprises points of extraction of the biogas as well.

The storage 400 allows to store the sewage 16 that, being already digested, gives a little contribution to the production of biogas 18 in the digester 4; the volume cleared in this way in the digestion chamber 40 allows the inward flow of fresh homogenised sewage 12, in order to maintain proper digestion conditions.

In the storage 400 the digested sewage 16, having a residual organic load, nevertheless undergoes a further digestion, which is, however, residual and involves a low production of biogas.

In the example, the storage 400 comprises a second bag-like casing 500 of flexible material and fluid-tight; the second casing 500 is entirely similar (or even identical) to the first casing 5 of the digester 4.

The second bag 500 is closed and defines, in an internal zone, a storage chamber 440 for the digested sewage 16.

The second bag 500 is hydraulically connected with the first bag 5, to allow the transfer of digested sewage 16 from the digester 4 (i.e. from the first casing 5) to the storage 400.

As schematically illustrated in FIGS. 22 and 23, the second bag 500 receives the digested sewage 16 through a piping 640 which connects it to the first bag 5; for example, the piping 640 connects the outlet pit 60 of the digester 4 to an inlet end 38t in the storage 400.

As for the first bag 5, one or more outlet points, through which the digested sewage 16 is removed from the second bag 500, are provided. In the example, a bottom wall of the second bag 500 has an opening or pit 560 which is connected to an unload pipe 561.

The second bag 500 comprises also one or more ports 57 of extraction of biogas 18; the ports 57 are made in the top wall 51 of the second bag 500 and are connected to respective extraction tubes 71, which in turn are connected to a header 710 to which also the extraction tubes 71 of the digester 4 are connected.

The second bag 500 comprises one or more safety valves 49 and hermetic hatches 56 (or manhole covers) for accessing the storage chamber 440.

In the example the storage 400 is devoid of stirrers and heater.

The second bag 500 can be used to carry out a second digestion stage. In other words, the first bag 5 and the second bag 500 compose a digester that operates according to two digestion stages in series. In this case, the second bag 500 is not a simple inactive storage, but instead it is an active part of the plant and allows to complete the digestion. The second bag 500 may then be provided with stirrers, heater and/or recirculation.

Both bags 5, 500, which for example are identical to each other, can operate at a constant level of sewage inside them.

The first bag 5 receives the feeding sewage 12 or fresh sewage, which undergoes a first digestion stage producing a biogas poor in methane and rich in carbon dioxide.

The composition of this biogas can vary greatly during a day, due to the change of internal conditions in the first bag 5 that takes place following the periodical injection of other feeding or fresh sewage.

The sewage 16 digested by the first bag 5 is received in the second bag 500 and undergoes a second digestion stage, which produces a biogas very rich in methane and poor in carbon dioxide.

This difference between the biogas produced by the first bag 5 and the biogas produced by the second bag 500 is related to the different concentrations and type of organic substance in the treated sewage and to the different operating conditions in the respective bag 5, 500.

The biogas 18 fuelled to the engine 81 is a mixture between the biogas produced by the first bag 5 and the biogas produced by the second bag 500. This allows to have a biogas composition that is less subject to fluctuations and having characteristics more suitable for use in the engine 81.

The digestion in two successive stages allows to exploit the energy potentiality of the available biomass at the best; for example, the digestion in two stages allows to reach up to an exploitation percentage of 95%, whereas the exploitation in a single-bag digester may be around 70%.

Furthermore, the digestion in two successive stages in two separate bags 5, 500 allows a better control of the concentration of nitrogen, which would stop the digestion process in case of high concentration.

In fact, in order to keep the nitrogen concentration below a threshold concentration capable of blocking the digestion, a dilution of the sewage in digestion 14 is required.

In the case of a plant with a single bag 5, the dilution of the sewage 14 could lead to a residence time too short for the sewage 14 itself and therefore to an incomplete digestion.

On the contrary, in the case of a plant with two bags 5, 500 in series, the dilution of the sewage 14 in the first bag 5 (which is the one where the production of nitrogen is higher and thus where the problem may occur) is performed using the digestate going out from the second bag 500. In other words, there is a recirculation of sewage from the second bag 500 to the first bag 5. For this purpose, a return line of sewage from the second bag-like casing 500 to the first bag-like casing 5 is provided; the return line includes an outlet pipe 61 for unloading sewage from the chamber 440 of the second bag 500 and an inlet pipe for introducing sewage into the digestion chamber 40 of the first bag 5.

An overall residence time adequate for a complete digestion is ensured by the additional residence volume given by the second bag 500.

Also the second bag 500 may have a system of recirculation and heating of the sewage as for the first bag 5. In particular, as shown in FIG. 33, a single heating exchanger 68 and a plurality of control valves (in particular solenoid valves 601, 602, 603, 604), which manage unloading from the first bag 5 and from the second bag 500 and re-introduction and recirculation of heated sewage in the bags 5, 500 themselves, may be provided.

In other words, the sewage recirculation line of the first bag 5 and the return line have a common length, on which the heat exchanger 68 is arranged. The influx of the unload pipes 61 of the first bag 5 and of second bag 500 into the common length is controlled by the solenoid valves 601, 602. The re-introduction of the heated and recirculated sewage into the first bag 5 and/or into the second bag 500 is controlled by the solenoid valves 603, 604.

In case, a third bag acting as a storage for the digestate coming out from the second bag 500 may be provided, similarly to the previously described storage 400.

A further embodiment of a co-generation installation 100 according to the present disclosure is schematically shown in FIG. 24, where the digester 4 is shown in a broken view to give greater prominence to the other components of the co-generation installation 100.

The header line 710 of the biogas 18 is provided with an overpressure and low-pressure valve 713, and a condensate separator 715; the header line 710 ends into a gasometer 717. The gasometer 717 is connected to a cooling section of the biogas, comprising a heat exchanger-cooler 701 connected to a refrigerator 703, a condensate separator 705, a blower 707 for the biogas. The blower 707 allows to raise the pressure of the biogas 18, which for example has a gauge pressure of 15 mbar in the bag 5, up to a pressure sufficient for feeding the engine 81 and/or the gasometer 717. A bypass circuit 702 to bypass the heat exchanger-cooler 701 can be provided.

The biogas circuit has a splitting, from which a first branch 72 goes to the co-generator 8 and a second branch 720 goes to a safety flare stack 723. The biogas circuit is provided with appropriate shut-off valves, including shut-off valve 725 and vent valve mounted on a container 800, in which the co-generator 8 is located. The container 800 is also provided with a gas leak sensor 805 and an exhaust tube 810 for the flue gas, in which an electric fan 820 is mounted.

A circuit 87 of recovery and reuse of heat includes the cooling circuit 83 of the engine 81, a pipe 42 for feeding the heating water, the heating coil 45 and/or the heat exchanger 68, a pipe 43 for returning the heating water.

In a different embodiment, schematically shown in FIG. 34, the header line 710 of the biogas 18 is provided with a biogas cooling section, comprising a heat exchanger-cooler 701 connected to a refrigerator 703, a condensate separator 705, a blower 707 for the biogas. A pressure adjuster 709, which adjusts the pressure of the biogas 18 fed to the engine 81 so as to ensure a regular operation of the engine 81 itself, is also provided. In fact, it should be considered that the output pressure from the blower 707 can have fluctuations, due to the fact that the pressure of the biogas produced by the plant varies during a day. The pressure adjuster 709 is used to fuel the engine 81 with a biogas 18 having a substantially constant pressure.

Between the blower 707 and the pressure adjuster 709, a pressure relief valve 708 connected to a return piping 708a is positioned, which discharges into the bag 5 (and/or in the second bag 500) any excess of biogas 18 that cannot be used by the engine 81.

In other words, in the case where the flow of biogas 18 sucked from the header line 710 is greater than the flow that can be used as a fuel by the engine 81 without alteration of the working speed rate of the engine 81 itself, the excess flow of biogas 18 (i.e. the flow which exceeds a current utilization capacity by the engine 81) is sent back to the bag 5, 500 through the return piping 708a. The excess of biogas 18 causes an overpressure in the biogas line, upstream of the pressure relief valve 708, which opens and discharges the excess in the return piping 708a.

Due to its large volume and the consequent capability of receiving a large amount of biogas with a small pressure change, the bag 5, 500 acts as a homogeniser and equalizer of the quantity and quality of the biogas 18 which is product over a day, allowing regular fuelling and operation of the engine 81.

Even the pipes for biogas connected to the bag-like casing 5, 500 may comprise flexible portions 700 to follow the deformations of the bag-like casing during the loading/unloading of the same and its shape changes in general.

A dimensional example of the plant 1 is given below with reference to FIGS. 29 and 30:
the length L5 of the bag 5 is 43 m;
the width P5 of the bag 5 is 23 m;
the bottom surface 91a of the pit 91 is tilted towards the centre: the height difference H91 between the edges and the centre is 0.375 m;
the length L91 of the bottom surface 91a is 37.5 m;
the width P91 of the bottom surface 91a is 17.5 m;
the length L96 between the outer edges of the containment beads 96 is 25 m;
the width P96 between the outer edges of the containment beads 96 is 45 m;
the overall length L96a of the area occupied by the bags, including the containment beads 96, is 48.5 m;
the overall width P96a of the area occupied by bags, including the containment beads 96, is 57 m.

The subject of the present disclosure has been described hitherto with reference to preferred embodiments thereof. It is understood that other embodiments relating to the same inventive idea may exist, all of these falling within the scope of protection of the claims which are provided hereinbelow.

1. A plant (1) for production of biogas (18), the plant (1) comprising a digester (4) including a bag-like casing (5) of flexible material and fluid-tight, said bag-like casing (5) being adapted for containing a sewage (14), wherein said bag-like casing (5) defines, in an internal zone, a digestion chamber (40) adapted for a digestion of the sewage (14), the plant (1) further comprising a heater (45, 68) for heating the sewage (14).

2. The plant (1) according to claim 1, wherein the heater comprises a heat exchanger (68) arranged on a sewage recirculation line (65), said recirculation line (65) comprising an outlet piping (61) to let sewage out of the digestion chamber (40) and an inlet piping (38a, 38p) to let sewage into the digestion chamber (40), the heat exchanger (68) being positioned between the outlet piping (61) and the inlet piping (38a, 38p, 38d).

3. The plant (1) according to claim 2, the inlet piping (38p, 38d) being configured to let sewage in on a free surface of the sewage (14) in the digestion chamber (40).

4. The plant (1) according to claim 2, the inlet piping (38p, 38d) comprising a main tube (38p) and a plurality of branches (38d), wherein the main tube (38p) is arranged around the perimeter of the bag-like casing (5) and each branch (38d) is configured to introduce sewage into the digestion chamber (40) in parallel with a respective flank of the bag-like casing (5).

5. The plant (1) according to claim 4, wherein the branches (38d) have inlet directions which are concordant with each other.

6. The plant (1) according to claim 1, comprising a second bag-like casing (500) of flexible material and fluid-tight, wherein said second bag-like casing (500) defines, in an internal zone, a chamber (440) for a sewage (16), said second bag-like casing (500) being hydraulically connected with the first bag-like casing (5) of the digester (4) in order to transfer sewage (16) from the first bag-like casing (5) to the second bag-like casing (500).

7. The plant (1) according to claim 6, wherein the second bag-like casing (500) is configured to carry out a second digestion stage of the sewage contained in the chamber (440), the first bag-like casing (5) and the second bag-like casing (500) composing a digester which works according to two digestion stages in series.

8. The plant (1) according to claim 6, comprising a return line for returning sewage from the second bag-like casing (500) to the first bag-like casing (5).

9. The plant (1) according to claim 8, wherein the heater comprises a heat exchanger (68) arranged on a sewage recirculation line (65), said recirculation line (65) comprising an outlet piping (61) to let sewage out of the digestion chamber (40) and an inlet piping (38a, 38p) to let sewage into the digestion chamber (40), the heat exchanger (68) being positioned between the outlet piping (61) and the inlet piping (38a, 38p, 38d), wherein said return line has a length which is in common with said recirculation line, the heat exchanger (68) being arranged on said common length.

10. The plant (1) according to claim 1, comprising a heater (45) positioned inside the digestion chamber (40).

11. The plant (1) according to claim 10, wherein the heater comprises a coil pipe (45) and a connection piping (42a, 43a) to connect the coil pipe (45) to a heating system (8) outside the bag-like casing (5), wherein a length of the connection piping (42a, 43a) inside the digestion chamber (40) comprises at least one flexible portion (421).

12. The plant (1) according to claim 1, wherein the bag-like casing (5) has a bottom face (52) resting on a first surface (91a) of a compartment (91) for at least partially receiving the bag-like casing (5), wherein the plant (1) comprises a heater (45) positioned between the bag-like casing (5) and the first surface (91a) and/or the lateral surface (91b) of the compartment (91), wherein the heater comprises a coil pipe (45) peripherally arranged around the lateral face (53) of the bag-like casing (5).

13. The plant (1) according to claim 1, wherein the digester (4) comprises at least one stirrer (55) for stirring the sewage (14), said at least one stirrer (55) being a submerged stirrer which is positioned inside the digestion chamber (40).

14. The plant (1) according to claim 1, wherein said digestion chamber (40) has a volume comprised between 1000 cubic meters and 5000 cubic meters.

15. A co-generation installation (100) comprising a plant (1) for production of biogas (18) according to claim 1, a co-generator (8) and fuelling means (71, 72) for fuelling the co-generator (8) with biogas (18), said fuelling means comprising a biogas line (71, 72) between the plant (1) and the co-generator (8).

16. The co-generation installation (100) according to claim 15, wherein the co-generator (8) comprises a heat recovery circuit (87) which is connected with the heater (45, 68).

17. The co-generation installation (100) according to claim 16 when depending at least on claim 2, wherein said heat exchanger (68) is configured to carry out a heat exchange between a recirculated sewage and the heat recovery circuit (87).

18. The co-generation installation (100) according to claim 15, wherein said biogas line (71, 72) between the plant (1) and the co-generator (8) comprises a pressure relief valve (708) connected to a return piping (708a) to return an excess of biogas (18), which exceeds a utilization capacity of the co-generator (8), to the bag-like casing (5) and/or to the second bag-like casing (500).
